(19) United States
(12) Patent Application Publication
(10) Pub. No.: US 2020/0124600 A1
(43) Pub. Date: Apr. 23, 2020

(54) METHODS OF TREATING AUTOIMMUNE AND INFLAMMATORY DISEASES

(71) Applicant: Genentech, Inc., South San Francisco, CA (US)

(72) Inventors: Michael TOWNSEND, South San Francisco, CA (US); Jason HACKNEY, South San Francisco, CA (US); Nandhini RAMAMOORTHI, South San Francisco, CA (US)

(73) Assignee: Genentech, Inc., South San Francisco, CA (US)

(21) Appl. No.: 16/579,404

(22) Filed: Sep. 23, 2019

Related U.S. Application Data
(63) Continuation of application No. PCT/US2018/023986, filed on Mar. 23, 2018.
(60) Provisional application No. 62/476,406, filed on Mar. 24, 2017.

(51) Int. Cl. G01N 33/564; A61K 31/4985; C12Q 1/6883; A61P 37/00; C07K 16/28

(52) U.S. Cl. CPC G01N 33/564; A61K 31/4985; C12Q 1/6883; A61P 37/00; A61K 2039/505; G01N 2800/52; G01N 2800/24; C12Q 2600/158; C07K 16/2887

(57) ABSTRACT

Provided herein are biomarkers and therapies for the treatment of autoimmune and/or inflammatory diseases, such as lupus, and methods of using BTK inhibitors. In particular, provided are biomarkers for patient selection and prognosis in lupus, as well as methods of therapeutic treatment, articles of manufacture and methods for making them, diagnostic kits, methods of detection and methods of advertising related thereto.

### CROSS-REFERENCE TO RELATED APPLICATIONS

This application is a continuation of International Application No. PCT/US2018/023986, filed Mar. 23, 2018, which claims priority to U.S. Provisional Application No. 62/476,406 filed Mar. 24, 2017, each of which is incorporated herein by reference in its entirety.

### FIELD

Provided herein are biomarkers and therapies for the treatment of autoimmune and inflammatory diseases, and method of using BTK inhibitors. In particular, provided are biomarkers for patient selection and prognosis in autoimmune and inflammatory diseases, as well as methods of therapeutic treatment, articles of manufacture and methods for making them, diagnostic kits, methods of detection and methods of advertising related thereto.

### BACKGROUND

Autoimmune and inflammatory diseases and disorders remain a significant threat to human health. Despite the significant advancement in the treatment of autoimmune and inflammatory diseases and disorders, improved therapies are still being sought. Many autoimmune and inflammatory diseases exhibit evidence of heterogeneity. For example, systemic lupus erythematosus (SLE) is a disease with evidence of heterogeneity in SLE patient populations. See Kennedy et al., Lupus Sci. & Med., 2015; 2:e000080. In view of this heterogeneity, a need exists for, in addition for new methods of treating autoimmune and inflammatory diseases (e.g., SLE), methods of identifying certain patients using diagnostic biomarkers which may improve treatment outcomes.

Plasmablasts are rapidly dividing, short-lived antibody secreting cells. Increases in plasmablasts have been identified in juvenile lupus patient blood, and increased abundance of antibody trancripts in lupus patients in general. E. Arce et al., J. Immunol. 167, 2361-2369 (2001); L. Bennett et al., J. Exp. Med. 197, 711-723 (2003). While plasmablasts represent a small proportion of B cells in the blood, they are responsible for the majority of antibody transcripts found in whole blood mRNA.

Protein kinases, the largest family of human enzymes, encompass well over 500 proteins. Bruton's Tyrosine Kinase (BTK) is a member of the Tec family of tyrosine kinases, and is a regulator of early B-cell development as well as mature B-cell activation, signaling, and survival. Evidence for the role of BTK in allergic disorders and/or autoimmune disease and/or inflammatory disease has been established in BTK-deficient mouse models. For example, in standard murine preclinical models of SLE, BTK deficiency has been shown to result in a marked amelioration of disease progression. Moreover, BTK deficient mice can also be resistant to developing collagen-induced arthritis and can be less susceptible to Staphylococcus-induced arthritis. A large body of evidence supports the role of B cells and the humoral immune system in the pathogenesis of autoimmune and/or inflammatory diseases. See, e.g., WO 2012/118750. Protein-based therapeutics (such as Rituxan) developed to deplete B cells, represent an approach to the treatment of a number of autoimmune and/or inflammatory diseases. Because of BTK's role in B-cell activation, inhibitors of BTK can be useful as inhibitors of B-cell mediated pathogenic activity (such as autoantibody production). BTK is also expressed in osteoclasts, mast cells and monocytes and has been shown to be important for the function of these cells. For example, BTK deficiency in mice is associated with impaired IgE-mediated mast cell activation (marked diminution of TNF-alpha and other inflammatory cytokine release), and BTK deficiency in humans is associated with greatly reduced TNF-alpha production by activated monocytes.

Inhibition of BTK activity can be useful for the treatment of allergic disorders and/or autoimmune and/or inflammatory diseases such as: SLE, rheumatoid arthritis, multiple vasculitides, idiopathic thrombocytopenic purpura (ITP), myasthenia gravis, allergic rhinitis, and asthma (Di Paolo et al (2011) Nature Chem. Biol. 7(1):41-50; Liu et al (2011) Jour. of Pharm. and Exper. Ther. 338(1):154-163). Specific BTK inhibitors have been reported (Liu (2011) Drug Metab. and Disposition 39(10):1840-1849; U.S. Pat. No. 7,884,108, WO 2010/056875; U.S. Pat. Nos. 7,405,295; 7,393,848; WO 2006/053121; U.S. Pat. No. 7,947,835; US 2008/0139557; U.S. Pat. No. 7,838,523; US 2008/0125417; US 2011/0118233; PCT/US2011/050034 “PYRIDINONES/PYRAZINONES, METHOD OF MAKING, AND METHOD OF USE THEREOF”, filed 31 Aug. 2011; PCT/US2011/050013 “PYRIDAZINONES, METHOD OF MAKING, AND METHOD OF USE THEREOF”, filed 31 Aug. 2011; U.S. Ser. No. 13/102,720 “PYRIDONE AND AZA-PYRIDONE COMPOUNDS AND METHODS OF USE”, filed 6 May 2011).

U.S. Pat. No. 8,716,274 (incorporated by reference herein in its entirety) discloses classes of heteroaryl pyridine and aza-pyridone compounds useful for inhibiting BTK. Compound (A) depicted below is one particular BTK inhibitor compound:

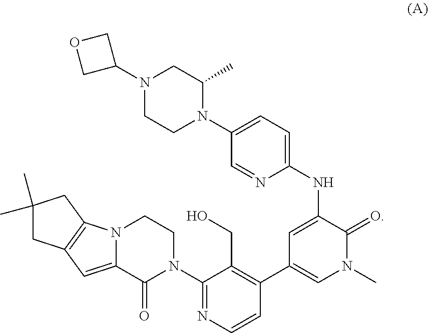 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1]

Compound (A) is: (S)-2-(3′-(hydroxymethyl)-1-methyl-5-((5-(2-methyl-4-(oxetan-3-yl)piperazin-1-yl)pyridin-2-yl)amino)-6-oxo-1,6-dihydro-[3,4′-bipyridin]-2′-yl)-7,7-dimethyl-2,3,4,6,7,8-hexahydro-1H-cyclopenta[4,5]pyrrolo[1,2-a]pyrazin-1-one. The chemical structure predominates in the case of any inconsistency between the chemical structure and the chemical name.

All references cited herein, including patent applications and publications, are incorporated by reference in their entirety.

### SUMMARY

Provided herein is a method for treating an individual with an autoimmune or inflammatory disease, comprising administering a therapeutically effective amount of a BTK inhibitor to the individual, wherein a sample from the individual has been found to have elevated levels of one or more biomarkers selected from the group consisting of IgJ, Mzb1, and Txndc5.

Also provided herein is a method for treating an autoimmune or inflammatory disease in an individual, the method comprising:
(a) determining that a sample from the individual comprises elevated levels of one or more biomarkers selected from the group consisting of IgJ, Mzb1, and Txndc5; and
(b) administering an effective amount of a BTK inhibitor to the individual, whereby the immunological disease or disorder is treated.

Also provided herein is a method for selecting a therapy for an individual with an autoimmune or inflammatory disease comprising determining levels of one or more biomarkers selected from the group consisting of IgJ, Mzb1, and Txndc5; and selecting a medicament based on the levels of the biomarkers.

Also provided herein is a method of identifying an individual having an autoimmune or inflammatory disease who is more or less likely to exhibit benefit from treatment comprising a BTK inhibitor by determining levels of one or more biomarkers selected from the group consisting of IgJ, Mzb1, and Txndc5 in a sample from the individual, wherein elevated levels of the biomarkers in the sample indicates that the individual is more likely to exhibit benefit from treatment comprising the BTK inhibitor or a reduced levels of the biomarkers indicates that the individual is less likely to exhibit benefit from treatment comprising the BTK inhibitor.

Also provided herein is an assay for identifying an individual with an autoimmune or inflammatory disease to receive a BTK inhibitor, the method comprising:
(a) determining levels of one or more biomarkers selected from the group consisting of IgJ, Mzb1, and Txndc5 in a sample from the individual; and
(b) recommending administration of a BTK inhibitor based upon the levels of the biomarkers.

Also provided herein is a diagnostic kit comprising one or more reagent for determining levels of one or more biomarkers selected from the group consisting of IgJ, Mzb1, and Txndc5 in a sample from an individual with an autoimmune or inflammatory disease, wherein detection of elevated levels of the biomarkers means increased efficacy when the individual is treated with a BTK inhibitor, and wherein detection of a low or substantially undetectable levels of a biomarker means a decreased efficacy when the individual with the autoimmune or inflammatory disease is treated with the BTK inhibitor.

In some embodiments of a method provided herein, the method further comprises administering an effective amount of the BTK inhibitor to the individual.

In some embodiments of a method, assay and/or kit provided herein, the sample is a blood sample.

In some embodiments of a method, assay and/or kit provided herein, the BTK inhibitor is an antibody, binding polypeptide, small molecule, and/or polynucleotide.

In some embodiments of a method, assay and/or kit provided herein, the BTK inhibitor is a small molecule. In some embodiments, the small molecule BTK inhibitor is Compound (A) or a pharmaceutically acceptable salt thereof.

In some embodiments of a method, assay and/or kit provided herein, the autoimmune or inflammatory disease is systemic lupus erythematosus. In some embodiments, the autoimmune or inflammatory disease is lupus nephritis. In some embodiments, the autoimmune or inflammatory disease is extra-renal lupus.

Biological markers and methods of their use for predicting response to treatment with B-cell antagonists (e.g, anti-CD20 antibodies) in autoimmune diseases such as rheumatoid arthritis, multiple sclerosis and lupus have been previously disclosed, but not the present plasmablast gene signature, and not with respect to BTK inhibition. See WO 2012/118750, the entire contents of which are hereby incorporated by reference.

As provided herein, transcriptional profiling of B-cell subsets identified a gene expression signature specific to plasmablasts. This signature is highly correlated with plasmablast abundance in an in vitro spike in experiment. Using FACS analysis of B-cell subsets in SLE patients, paired with RNA-sequencing, the present gene expression signature showed strong correlation with the frequency of plasmablasts in whole blood. While plasmablasts represent a small proportion of B-cells in the blood, they are responsible for the majority of antibody transcripts found in whole blood mRNA. Expanding to two additional phase II clinical trial cohorts, it was found that the plasmablast signature was correlated with disease activity using the SLEDAI disease activity index. This association was driven by correlation of plasmablasts with presence of anti-DNA antibodies, low levels of complement and leukopenia. Increased plasmablast signature was also associated with high levels of interferon activity. Patient race/ethnicity was also predictive of plasmablast signature levels, independent of disease severity. Standard of care medications, particularly mycophenolate, reduced the expression of plasmablast marker genes. Treatment of patients with rituximab, for example, lead to a profound, though ultimately transient, decrease in plasmablast signature expression.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-1 and 1A-2. Plasmablast differentiation in vitro and sorting strategy. Plasmablasts were differentiated from CD20⁺CD27⁺ memory B cells under culture conditions containing CpG for 7 days along with cytokines IL-2, IL-6, IL-10, IL-15, IFNα. Naïve B cells (CD20⁺CD27⁻), and FACS sorted CD20⁺CD27⁺ activated B cells and differentiated CD20^{lo}CD38⁺ plasmablasts were used for gene expression profiling.

FIG. 1B. Heatmap of genes specifically expressed by plasmablasts. Genes that were more highly expressed by plasmablasts than naive B cells and activated B cells by at least 10-fold, at an FDR of 0.001, and had an expression level >5 RPKM in plasmablasts were identified. Values represent the variance stabilized data that has been standardized to mean 0, standard deviation of 1 within each gene.

FIG. 2A. Heatmap of candidate plasmablast signature genes in PBMC samples into which increasing numbers of plasmablasts were added. Plasmablasts were spiked into PBMCs from two separate donors, as indicated in black and grey above the heatmap. Values represent the ΔCt of each gene relative to HPRT1, and standardized to a mean of 0 and a standard deviation of 1.

FIGS. 2B-1, 2B-2, 2B-3 and 2B-4. Expression levels of plasmablast signature genes, relative to HPRT1, or the mean of all three genes, compared to the percent of plasmablasts present in each sample. Dotted and dashed lines indicate the different PBMC donors, while different symbols represent the different donors for plasmablasts. Linear regression analysis was used to predict the expression of the plasmablast signature, or component genes, incorporating PBMC donor and plasmablast donor into the model. All four models were highly statistically significant, with p<1×10⁻¹⁰. The predictive power of the model was reported as the r² from the linear model.

FIGS. 2C-1, 2C-2, 2C-3 and 2C-4. Relative expression of plasmablast genes to HPRT1 or the mean of all three signature genes measured in B cell populations isolated from healthy donors one week after receiving flu vaccine. N=naïve B cells, M=memory B cells, PB=plasmablasts. Plasmablasts have the highest expression of marker genes compared to the other populations. Stars indicate the statistical significance of the differences between the B cell populations, using linear regression, including the donor as a covariate; *=p<0.05, **=p<0.01, ***=p<0.001.

FIGS. 2D-1, 2D-2, 2D-3 and 2D-4. Plasmablast signature and component genes are correlated with frequency of plasmablasts measured by FACS in lupus patient blood. Ig CD19⁺CD27⁺⁺CD38⁺⁺ plasmablasts were measured as a percent of whole blood cells in 43 patients over as many as 3 time points, for which we had accompanying RNA-sequencing data, for a total of 96 samples. Gene expression values are presented as the RPKM of individual genes, or the geometric mean RPKM for the three gene signature. Correlation coefficients were calculated using Spearman's rank-order method.

FIG. 3A. Plasmablast signature correlates with disease activity measured by SLEDAI. Values represent the mean expression of plasmablast signature genes relative to HPRT1. SLEDAI and plasmablast signature values are from samples collected prior to initiation of treatment. Correlation coefficient was determined using Spearman's rank-order method.

FIGS. 3B-1, 3B-2 and 3B-3. Individual components of the SLEDAI composite index are associated with increased expression of plasmablast signature genes. Linear regression was used to assess the statistical significance between patients that exhibited each of the symptoms and those that did not; stars indicate the significance level in this test: *=p<0.05, **=p<0.01, ***=p<0.001.

FIGS. 3C-1, 3C-2 and 3C-3. Serum C3 and C4 complement levels and serum anti-dsDNA antibody titers correlate with plasmablast signature expression. Correlation coefficients were calculated using Spearman's rank-order method.

FIGS. 3D-1 and 3D-2. Whole blood interferon signature expression (ISM) correlates with plasmablast signature values. Correlation coefficients were calculated using Spearman's rank-order method.

FIG. 4A. Treatment of patients with rituximab decreases plasmablast signature expression levels. Lines indicate the mean expression level within the rituximab treated cohort (dashed line) or the placebo cohort (solid line), with error bars indicating the standard error of the mean. Black arrows indicate when patients received infusions of drug or placebo. Expression of plasmablast signature genes was modeled using a linear mixed effects model, incorporating age, race/ethnicity, concomitant medication used, interferon activity, SLEDAI, and treatment arm and time point and their interaction as fixed effects, and patient as a random effect. Red stars indicate time points that significantly differed specifically in the rituximab-treated arm: *=p<0.05, **=p<0.01, ***=p<0.001.

FIG. 4B. Treatment with mycophenolate and rituximab independently decrease plasmablast signature expression levels. Lines indicate the mean of the placebo cohort (solid line) or rituximab-treated cohort (dashed line), with standard error of the mean indicated by error bars. Arrows indicate when patients received infusions of placebo or rituximab. Expression values were modeled using linear mixed effects model incorporating age, interferon activity and treatment arm and visit and their interaction, with patient as a random effect. Stars at the top of the graph indicate time points where rituximab-treated patients showed a significant reduction from baseline beyond the placebo arm, while stars near the bottom of the graph indicate time points that differed from baseline regardless of treatment: *=p<0.05, **=p<0.01, ***=p<0.001.

FIG. 4C. Patients that had detectable anti-chimeric antibody (HACA) have higher expression of plasmablast marker genes. Lines indicate the mean expression of plasmablast marker genes in rituximab treated patients that had detectable HACA (solid line), or those that never had detectable HACA (dashed line), error bars indicate the standard error of the mean.

FIG. 5A. Patients treated with mycophenolate mofetil (MMF) or methotrexate (MTX) show lower plasmablast expression than patients treated with azathioprine (AZA). Screening plasmablast signature values were compared between patients on different immunosuppressive regimes. Statistical significance was tested using linear regression, comparing AZA to each of the other two treatments. Stars indicate significant differences between treatments: *=p<0.05, **=p<0.01, ***=p<0.001.

FIG. 5B. Patients on MMF treatment at screening trend toward having lower plasmablast signature than those that were not on MMF treatment. The p-value was calculated using linear regression between patients taking MMF and those not taking MMF at their screening visit.

FIG. 6A. Patients with European ancestry have lower levels of plasmablast expression than other ethnicities in the EXPLORER clinical trial cohort. Values represent mean expression levels of plasmablast genes relative to HPRT1. Screening visit values were compared across self-reported race/ethnicity using linear regression. Stars indicate significance of differences compared to patients self-reporting as White/Caucasian: *=p<0.05, **=p<0.01, ***=p<0.001.

FIG. 6B. LUNAR patients show no significant differences based on race/ethnicity. Using linear regression, no significant differences were observed across ethnicities.

FIG. 6C. Patients with European ancestry show lower expression of plasmablast markers in the ROSE clinical trial cohort. Values represent geometric mean RPKM of plasmablast genes. Screening visit values were compared across self-reported race/ethnicity using linear regression against the log2-transformed mean RPKM. Stars indicate significance of differences compared to patients self-reporting as White/Caucasian: *=p<0.05, **=p<0.01, ***=p<0.001.

FIG. 7. Dose response curves for inhibition of CD4OL mediated plasmablast differentiation by BTK inhibitor GDC-0852 show inhibition of plasmablast differentiation in a dose dependent manner. Percentages of plasmablasts in four healthy donors was determined using FACS analysis to calculate an IC50 value for each donor.

FIGS. 8A, 8B, 8C and 8D. BTK inhibition reduces the plasmablast gene signature. Memory B cells from 4 healthy donors were differentiated into plasmablasts using conditions described above, in the presence of DMSO vehicle or 370 nM GDC-0852. Expression of plasmablast signature genes was measured by Fluidigm, and normalized to a housekeeping gene (HPRT1). Expression values are plotted as the relative transcript abundance to the housekeeping gene. The plasmablast signature was calculated as the geometric mean of the relative abundances of the three individual genes.

FIG. 9. Plasmablast gene expression correlates with plasmablast cell numbers. Percentages of plasmablasts as determined by FACS analysis correlate with the plasmablast signature, as determined in FIG. 8D (Spearman rho=0.81). White points indicate samples differentiated in the presence of DMSO, while black points represent GDC-0852-treated samples.

FIG. 10. BTK inhibitor GDC-0852 inhibited CpG mediated plasmablast differentiation in a dose dependent manner Percentages of plasmablasts in four healthy donors was determined using FACS analysis to calculate an IC50 value for each donor.

### DETAILED DESCRIPTION

### I. Definitions

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), “(O)NR₂ (“amidate”), P(O)R, P(O)OR′, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “primer” refers to a single stranded polynucleotide that is capable of hybridizing to a nucleic acid and following polymerization of a complementary nucleic acid, generally by providing a free 3′-OH group.

The term “small molecule” refers to any molecule with a molecular weight of about 2000 daltons or less, preferably of about 500 daltons or less.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

A “blocking” antibody or an “antagonist” antibody is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The term “detection” includes any means of detecting, including direct and indirect detection.

The term “biomarker” as used herein refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features. In some embodiments, a biomarker is a gene. Biomarkers include, but are not limited to, polynucleotides (e.g., DNA, and/or RNA), polypeptides, polypeptide and polynucleotide modifications (e.g. posttranslational modifications), carbohydrates, and/or glycolipid-based molecular markers.

The terms “biomarker signature,” “signature,” “biomarker expression signature,” or “expression signature” are used interchangeably herein and refer to one or a combination of biomarkers whose expression is an indicator, e.g., predictive, diagnostic, and/or prognostic. The biomarker signature may serve as an indicator of a particular subtype of a disease or disorder (e.g., cancer) characterized by certain molecular, pathological, histological, and/or clinical features. In some embodiments, the biomarker signature is a “gene signature.” The term “gene signature” is used interchangeably with “gene expression signature” and refers to one or a combination of polynucleotides whose expression is an indicator, e.g., predictive, diagnostic, and/or prognostic. In some embodiments, the biomarker signature is a “protein signature.” The term “protein signature” is used interchangeably with “protein expression signature” and refers to one or a combination of polypeptides whose expression is an indicator, e.g., predictive, diagnostic, and/or prognostic.

The “amount” or “level” of a biomarker associated with an increased clinical benefit to an individual is a detectable level in a biological sample. These can be measured by methods known to one skilled in the art and also disclosed herein. The expression level or amount of biomarker assessed can be used to determine the response to the treatment.

The terms “level of expression” or “expression level” in general are used interchangeably and generally refer to the amount of a biomarker in a biological sample. “Expression” generally refers to the process by which information (e.g., gene-encoded and/or epigenetic) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., posttranslational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

“Elevated expression,” “elevated expression levels,” or “elevated levels” refers to an increased expression or increased levels of a biomarker in an individual relative to a control, such as an individual or individuals who are not suffering from the disease or disorder (e.g., cancer) or an internal control (e.g., housekeeping biomarker).

“Reduced expression,” “reduced expression levels,” or “reduced levels” refers to a decrease expression or decreased levels of a biomarker in an individual relative to a control, such as an individual or individuals who are not suffering from the disease or disorder (e.g., cancer) or an internal control (e.g., housekeeping biomarker). In some embodiments, reduced expression is little or no expression.

In certain embodiments, the term “at the reference level” refers to a level of the biomarker in the sample from the individual or patient that is essentially identical to the reference level or to a level that differs from the reference level by up to 1%, up to 2%, up to 3%, up to 4%, up to 5%. In some embodiments, the reference level is the median level of the biomarker in a reference population. In some embodiments, a reference level of a marker is the mean level of the marker in a reference population. In some embodiments, a reference level of a marker is the average level of the marker in a reference population.

In certain embodiments, the term “above the reference level” refers to a level of the biomarker in the sample from the individual or patient above the reference level by at least 5%, 10%, 20%, 25%, 30%, 40%, 50%, 60%, 70%, 80%, 85%, 90%, 95%, 100% or greater, determined by the methods described herein, as compared to the reference level. In some embodiments, the reference level is the median level in a reference population. In some embodiments, a reference level of a marker is the mean level of the marker in a reference population.

In certain embodiments, the term “below the reference level” refers to a level of the biomarker in the sample from the individual or patient below the reference level by at least 5%, 10%, 20%, 25%, 30%, 40%, 50%, 60%, 70%, 80%, 85%, 90%, 95%, 100% or greater, determined by the methods described herein, as compared to the reference level. In some embodiments, the reference level is the median level in a reference population. In some embodiments, a reference level of a marker is the mean level of the marker in a reference population. In some embodiments, a reference level of a marker is the average level of the marker in a reference population.

The term “housekeeping biomarker” refers to a biomarker or group of biomarkers (e.g., polynucleotides and/or polypeptides) which are typically similarly present in all cell types. In some embodiments, the housekeeping biomarker is a “housekeeping gene.” A “housekeeping gene” refers herein to a gene or group of genes which encode proteins whose activities are essential for the maintenance of cell function and which are typically similarly present in all cell types.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The term “multiplex-PCR” refers to a single PCR reaction carried out on nucleic acid obtained from a single source (e.g., an individual) using more than one primer set for the purpose of amplifying two or more DNA sequences in a single reaction.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain biomarkers in a biological sample from an individual.

The term “sample,” as used herein, refers to a composition that is obtained or derived from a subject and/or individual of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. Samples include, but are not limited to, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

For the purposes herein a “section” of a tissue sample is meant a single part or piece of a tissue sample, e.g. a thin slice of tissue or cells cut from a tissue sample. It is understood that multiple sections of tissue samples may be taken and subjected to analysis, provided that it is understood that the same section of tissue sample may be analyzed at both morphological and molecular levels, or analyzed with respect to both polypeptides and polynucleotides.

By “correlate” or “correlating” is meant comparing, in any way, the performance and/or results of a first analysis or protocol with the performance and/or results of a second analysis or protocol. For example, one may use the results of a first analysis or protocol in carrying out a second protocols and/or one may use the results of a first analysis or protocol to determine whether a second analysis or protocol should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endPoint indicating a benefit to the individual, including, without limitation, (1) inhibition, to some extent, of disease progression (e.g., cancer progression), including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e. reduction, slowing down or complete stopping) of metasisis; (5) relief, to some extent, of one or more symptoms associated with the disease or disorder (e.g., cancer); (6) increase in the length of progression free survival; and/or (9) decreased mortality at a given Point of time following treatment.

The term “substantially the same,” as used herein, denotes a sufficiently high degree of similarity between two numeric values, such that one of skill in the art would consider the difference between the two values to be of little or no biological and/or statistical significance within the context of the biological characteristic measured by said values (e.g., Kd values or expression). The difference between said two values is, for example, less than about 50%, less than about 40%, less than about 30%, less than about 20%, and/or less than about 10% as a function of the reference/comparator value.

The phrase “substantially different,” as used herein, denotes a sufficiently high degree of difference between two numeric values such that one of skill in the art would consider the difference between the two values to be of statistical significance within the context of the biological characteristic measured by said values (e.g., Kd values). The difference between said two values is, for example, greater than about 10%, greater than about 20%, greater than about 30%, greater than about 40%, and/or greater than about 50% as a function of the value for the reference/comparator molecule.

The word “label” when used herein refers to a detectable compound or composition. The label is typically conjugated or fused directly or indirectly to a reagent, such as a polynucleotide probe or an antibody, and facilitates detection of the reagent to which it is conjugated or fused. The label may itself be detectable (e.g., radioisotope labels or fluorescent labels) or, in the case of an enzymatic label, may catalyze chemical alteration of a substrate compound or composition which results in a detectable product.

An “effective amount” of an agent refers to an amount effective, at dosages and for periods of time necessary, to achieve the desired therapeutic or prophylactic result.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject., A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or to slow the progression of a disease.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs of this invention include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use in this invention include, but are not limited to, those chemotherapeutic agents described above.

An “individual” or “subject” is a mammal Mammals include, but are not limited to, domesticated animals (e g., cows, sheep, cats, dogs, and horses), primates (e.g., humans and non-human primates such as monkeys), rabbits, and rodents (e.g., mice and rats). In certain embodiments, the individual or subject is a human.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain embodiments, the manufacture or kit is promoted, distributed, or sold as a unit for performing the methods described herein.

The phrase “based on” when used herein means that the information about one or more biomarkers is used to inform a treatment decision, information provided on a package insert, or marketing/promotional guidance, etc.

As is understood by one skilled in the art, reference to “about” a value or parameter herein includes (and describes) embodiments that are directed to that value or parameter per se. For example, description referring to “about X” includes description of “X”.

It is understood that aspect and embodiments described herein include “consisting” and/or “consisting essentially of” aspects and embodiments. As used herein, the singular form “a”, “an”, and “the” includes plural references unless indicated otherwise.

### II. Methods and Uses

Provided herein are methods utilizing a plasmablast biomarker. In particular, methods utilizing a BTK inhibitor and a plasmablast biomarker. For example provided are methods for treating an individual with disease or disorder comprising administering a therapeutically effective amount of a BTK inhibitor to the individual if the individual has been found to have presence and/or elevated levels of a plasmablast biomarker. Further provided herein are methods for treating a disease or disorder in an individual, the method comprising: determining that a sample from the individual comprises elevated levels of a plasmablast biomarker, and administering an effective amount of a BTK inhibitor to the individual, whereby the disease or disorder is treated. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

Provided herein are methods of treating a disease or disorder in an individual comprising administering to the individual an effective amount of a BTK inhibitor, wherein treatment is based upon presence and/or elevated levels of a plasmablast biomarker in a sample from the individual. In some embodiments, the plasmablast biomarker is expression of one or more of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

In addition, provided herein are methods for selecting a therapy for an individual with a disease or disorder comprising determining presence and/or levels of a plasmablast biomarker, and selecting a medicament based on the presence and/or levels of the biomarker. In some embodiments, the medicament is selected based upon elevated levels of the plasmablast biomarker. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

Provided herein are methods of identifying an individual with a disease or disorder who is more or less likely to exhibit benefit from treatment comprising a BTK inhibitor, the method comprising: determining presence and/or levels of a plasmablast biomarker in a sample from the individual, wherein the presence and/or elevated levels of the plasmablast biomarker in the sample indicates that the individual is more likely to exhibit benefit from treatment comprising the BTK inhibitor or absence and/or reduced levels of the plasmablast biomarker indicates that the individual is less likely to exhibit benefit from treatment comprising the BTK inhibitor. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

Provided herein are also assays for identifying an individual with a disease or disorder to receive a BTK inhibitor, the method comprising: (a) determining presence and/or levels of a plasmablast biomarker in a sample from the individual; (b) recommending a BTK inhibitor based upon the presence and/or levels of the plasmablast biomarker. In some embodiments, the BTK inhibitor is recommended based upon elevated levels of the plasmablast biomarker. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

Provided herein are diagnostic kits comprising one or more reagent for determining levels of a plasmablast biomarker in a sample from an individual with a disease or disorder, wherein detection of presence and/or elevated levels of the plasmablast biomarker means increased efficacy when the individual is treated with a BTK inhibitor, and wherein detection of a low or substantially undetectable levels of a plasmablast biomarker means a decreased efficacy when the individual with the disease is treated with the BTK inhibitor. Provided herein are also articles of manufacture comprising, packaged together, a pharmaceutical composition comprising a BTK inhibitor, and a package insert indicating that the BTK inhibitor is for treating a patient with a disease or disorder based on expression of a plasmablast biomarker. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

Further provided herein are methods for treating a disease or disorder in an individual comprising administering to the individual an effective amount of a BTK inhibitor, and assessing levels of one or more plasmablast biomarkers in a sample from the individual (e.g., compared to a reference) during treatment with the BTK inhibitor. Also provided are methods of treating a disease or disorder in an individual comprising administering to the individual an effective amount of a BTK inhibitor, wherein treatment is based upon levels of one or more plasmablast biomarkers in a sample from the individual (e.g., compared to a reference). Provided are methods of monitor responsiveness in an individual to treatment comprising a BTK inhibitor, the method comprising: determining levels of one or more plasmablast biomarkers in a sample from the individual, wherein reduced levels of one or more plasmablast biomarkers (e.g., compared to a reference) in the sample indicates that the individual is more likely responsive to treatment comprising the BTK inhibitor, or elevated levels and/or levels substantially the same as pretreatment levels of one or more plasmablast biomarkers (e.g., compared to a reference) indicates that the individual is less likely responsive to treatment comprising the BTK inhibitor. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

Additionally provided are methods of determining whether an individual with a disease or disorder should continue or discontinue treatment comprising a BTK inhibitor, the method comprising measuring in a sample from the individual levels of one or more plasmablast biomarkers, wherein elevated levels and/or levels substantially the same as pretreatment levels of one or more plasmablast biomarkers (e.g., compared to a reference) determines the individual should discontinue treatment comprising the BTK inhibitor and reduced levels of one or more plasmablast biomarkers (e.g., compared to a reference) determines the individual should continue treatment comprising the BTK inhibitor. In some embodiments, the plasmablast biomarker is selected from the group of gene signatures consisting of IgJ, Mzb1, and Txndc5. In some embodiments, gene expression of IgJ, Mzb1, and Txndc5 is polypeptide expression determined by measuring the level of mRNA for said gene in a patient's blood relative to a reference level. In some embodiments, the disease or disorder is an autoimmune or inflammatory disease or disorder. In some embodiments, the disease or disorder is SLE. In some embodiments, the disease or disorder is lupus nephritis. In some embodiments, the disease or disorder is extra-renal lupus.

In some embodiments, the method comprises: (a) measuring the RNA level of one, two, or three biomarkers selected from IgJ, TXNDC5 and MZB1 in a biological sample from the patient; (b) comparing the RNA level measured in (a) to a reference level; and (c) identifying the patient as more likely to benefit from BTK inhibitor therapy when the RNA level measured in (a) is above the reference level. In some embodiments, the RNA is mRNA. In some embodiments, the measuring the mRNA levels comprises amplification. In some embodiments, the measuring the mRNA levels comprises quantitative PCR. In some embodiments, the measuring the mRNA levels comprises amplifying the mRNA and detecting the amplified product, thereby measuring the level of the mRNA. In some embodiments, the reference level is the median level of the respective marker in a reference population.

In some embodiments, a reference level of a marker is the median level of the marker in a reference population. In any of the embodiments described herein, the reference level may be the mean level of the respective marker in a reference population. In some embodiments, a reference level of a marker is the average level of the marker in a reference population. Nonlimiting exemplary reference populations include patients with immune or inflammatory disease, healthy individuals, and a group including healthy individuals and patients with immune or inflammatory disease. In some embodiments, a reference population comprises patients with SLE.

In some embodiments, the method of analysis or detection of the biomarker has a p value that is less than 0.05. In some embodiments, the method has a specificity that is higher than 80%. In some embodiments, the method has a sensitivity that is higher than 80%. In some embodiments, the method has a ROC that is higher than 70%. In some embodiments, the method has an AUC that is higher than 70%. In some embodiments, the method has a positive predictive value that is higher than 70%. In some embodiments, the method has a negative predictive value that is higher than 70%. In some embodiments, said reference gene expression profile is from a subject in a reference population of patients and/or healthy volunteers. In some embodiments, the comparing step comprises at least one of: comparing digital images of the expression profiles and comparing databases of expression data.

In some embodiments of any of the above methods, the plasmablast biomarker is IgJ. In some embodiments of any of the above methods, the plasmablast biomarker is Mzb1. In some embodiments of any of the above methods, the plasmablast biomarker is Txndc5. In some embodiments of any of the above methods, the one or more plasmablast biomarkers is IgJ and Mzb1. In some embodiments of any of the above methods, the one or more plasmablast biomarkers is IgJ and Txndc5. In some embodiments of any of the above methods, the one or more plasmablast biomarkers is Txndc5 and Mzb1. In some embodiments of any of the above methods, the one or more plasmablast biomarkers is IgJ, Mzb1 and Txndc5.

In some of the above embodiments, the sample is a urine sample. In some embodiments, the sample is a blood sample. In some embodiments, the biological sample is selected from blood, serum, plasma, and peripheral blood mononucleocytes (PBMCs). In some embodiments, the biological sample is RNA obtained from blood, e.g., whole blood or a cellular fraction of blood, such as PBMC. In some embodiments, the biological sample is serum or plasma. The sample may be taken before treatment, during treatment or post-treatment. The sample may be taken from a patient who is suspected of having, or is diagnosed as having SLE or other immune or inflammatory disease, and hence is likely in need of treatment. Alternatively, the sample may be taken from a normal individual who is not suspected of having any disease. In some embodiments, RNA is extracted from a biological sample described herein prior to detecting or measuring the mRNA level of a marker.

Presence and/or expression levels/amount of a biomarker can be determined qualitatively and/or quantitatively based on any suitable criterion known in the art, including but not limited to DNA, mRNA, cDNA, proteins, protein fragments and/or gene copy number. In certain embodiments, presence and/or expression levels/amount of a biomarker in a first sample is increased as compared to presence/absence and/or expression levels/amount in a second sample. In certain embodiments, presence/absence and/or expression levels/amount of a biomarker in a first sample is decreased as compared to presence and/or expression levels/amount in a second sample. In certain embodiments, the second sample is a reference sample, reference cell, reference tissue, control sample, control cell, or control tissue. Additional disclosures for determining presence/absence and/or expression levels/amount of a gene are described herein.

In some embodiments of any of the methods, elevated expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

In some embodiments of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene or mRNA)), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

Presence and/or expression level/amount of various biomarkers in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (as for example Serum ELISA), biochemical enzymatic activity assays, in situ hybridization, Southern analysis, Northern analysis, whole genome sequencing, polymerase chain reaction (“PCR”) including quantitative real time PCR (“qRT-PCR”) and other amplification type detection methods, such as, for example, branched DNA, SISBA, TMA and the like), RNA-Seq, FISH, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments, presence and/or expression level/amount of a biomarker is determined using a method comprising: (a) performing gene expression profiling, PCR (such as rtPCR), RNA-seq, microarray analysis, SAGE, MassARRAY technique, or FISH on a sample (such as a subject cancer sample); and b) determining presence and/or expression level/amount of a biomarker in the sample. In some embodiments, the microarray method comprises the use of a microarray chip having one or more nucleic acid molecules that can hybridize under stringent conditions to a nucleic acid molecule encoding a gene mentioned above or having one or more polypeptides (such as peptides or antibodies) that can bind to one or more of the proteins encoded by the genes mentioned above. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like).

Samples from mammals can be conveniently assayed for mRNAs using Northern, dot blot or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member). Optionally, the sequence of the amplified target cDNA can be determined.

Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, a selection of genes whose expression correlates with increased or reduced clinical benefit of anti-angiogenic therapy may be arrayed on a solid support. Hybridization of a labeled probe with a particular array member indicates that the sample from which the probe was derived expresses that gene.

According to some embodiments, presence and/or expression level/amount is measured by observing protein expression levels of an aforementioned gene. In certain embodiments, the method comprises contacting the biological sample with antibodies to a biomarker described herein under conditions permissive for binding of the biomarker, and detecting whether a complex is formed between the antibodies and biomarker. Such method may be an in vitro or in vivo method. In one embodiment, an antibody is used to select subjects eligible for therapy with BTK inhibitor, e.g., a biomarker for selection of individuals.

In certain embodiments, the presence and/or expression level/amount of biomarker proteins in a sample is examined using IHC and staining protocols. IHC staining of tissue sections has been shown to be a reliable method of determining or detecting presence of proteins in a sample. In some embodiments of any of the methods, assays and/or kits, the plasmablast biomarker is selected from one or more of IgJ, Mzb1, and Txndc5. In some embodiments, IgJ, Mzb1, and/or Txndc5 is detected by immunohistochemistry. In some embodiments, elevated expression of a plasmablast biomarker in a sample from an individual is elevated protein expression and, in further embodiments, is determined using IHC. In one embodiment, expression level of biomarker is determined using a method comprising: (a) performing IHC analysis of a sample with an antibody; and b) determining expression level of a biomarker in the sample. In some embodiments, IHC staining intensity is determined relative to a reference. In some embodiments, the reference is a reference value. In some embodiments, the reference is a reference sample (e.g., control cell line staining sample). In some embodiments, the tissue is renal tissue. In other embodiments, the above techniques are performed using fluorescence in-situ hybridization in place of IHC.

IHC may be performed in combination with additional techniques such as morphological staining and/or fluorescence in-situ hybridization. Two general methods of IHC are available; direct and indirect assays. According to the first assay, binding of antibody to the target antigen is determined directly. This direct assay uses a labeled reagent, such as a fluorescent tag or an enzyme-labeled primary antibody, which can be visualized without further antibody interaction. In a typical indirect assay, unconjugated primary antibody binds to the antigen and then a labeled secondary antibody binds to the primary antibody. Where the secondary antibody is conjugated to an enzymatic label, a chromogenic or fluorogenic substrate is added to provide visualization of the antigen. Signal amplification occurs because several secondary antibodies may react with different epitopes on the primary antibody.

The primary and/or secondary antibody used for IHC typically will be labeled with a detectable moiety. Numerous labels are available which can be generally grouped into the following categories: (a) Radioisotopes, such as ³⁵S, ¹⁴C, ¹²⁵I, ³H, and ¹³¹I; (b) colloidal gold particles; (c) fluorescent labels including, but are not limited to, rare earth chelates (europium chelates), Texas Red, rhodamine, fluorescein, dansyl, Lissamine, umbelliferone, phycocrytherin, phycocyanin, or commercially available fluorophores such SPECTRUM ORANGE7 and SPECTRUM GREEN7 and/or derivatives of any one or more of the above; (d) various enzyme-substrate labels are available and U.S. Pat. No. 4,275,149 provides a review of some of these. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like.

Examples of enzyme-substrate combinations include, for example, horseradish peroxidase (HRPO) with hydrogen peroxidase as a substrate; alkaline phosphatase (AP) with para-Nitrophenyl phosphate as chromogenic substrate; and β-D-galactosidase (β-D-Gal) with a chromogenic substrate (e.g., p-nitrophenyl-β-D-galactosidase) or fluorogenic substrate (e.g., 4-methylumbelliferyl-β-D-galactosidase). For a general review of these, see U.S. Pat. Nos. 4,275,149 and 4,318,980.

In some embodiments of any of the methods, plasmablast biomarkers are detected by immunohistochemistry using a diagnostic antibody (i.e., primary antibody). In some embodiments, the tissue to be analyzed is renal tissue. In some embodiments, the diagnostic antibody specifically binds IgJ, Mzb1 or Txndc5. In some embodiments of any of the diagnostic antibodies, the diagnostic antibody is a nonhuman antibody. In some embodiments, the diagnostic antibody is a rat, mouse, or rabbit antibody. In some embodiments, the diagnostic antibody is a monoclonal antibody. In some embodiments, the diagnostic antibody is directly labeled.

In alternative methods, the sample may be contacted with an antibody specific for said biomarker under conditions sufficient for an antibody-biomarker complex to form, and then detecting said complex. The presence of the biomarker may be detected in a number of ways, such as by Western blotting and ELISA procedures for assaying a wide variety of tissues and samples, including plasma or serum. A wide range of immunoassay techniques using such an assay format are available, see, e.g., U.S. Pat. Nos. 4,016,043, 4,424,279 and 4,018,653. These include both single-site and two-site or “sandwich” assays of the non-competitive types, as well as in the traditional competitive binding assays. These assays also include direct binding of a labeled antibody to a target biomarker.

Presence and/or expression level/amount of a selected biomarker in a tissue or cell sample may also be examined by way of functional or activity-based assays. For instance, if the biomarker is an enzyme, one may conduct assays known in the art to determine or detect the presence of the given enzymatic activity in the tissue or cell sample.

In certain embodiments, the samples are normalized for both differences in the amount of the biomarker assayed and variability in the quality of the samples used, and variability between assay runs. Such normalization may be accomplished by detecting and incorporating the expression of certain normalizing biomarkers, including well known housekeeping genes, such as ACTB. Alternatively, normalization can be based on the mean or median signal of all of the assayed genes or a large subset thereof (global normalization approach). On a gene-by-gene basis, measured normalized amount of a subject sample mRNA or protein is compared to the amount found in a reference set. Normalized expression levels for each mRNA or protein per tested sample per subject can be expressed as a percentage of the expression level measured in the reference set. The presence and/or expression level/amount measured in a particular subject sample to be analyzed will fall at some percentile within this range, which can be determined by methods well known in the art.

In certain embodiments, relative expression level of a gene is determined as follows: Relative expression gene1 sample1=2 exp (Ct housekeeping gene−Ct gene1) with Ct determined in a sample.

Relative expression gene1 reference RNA=2 exp (Ct housekeeping gene−Ct gene1) with Ct determined in the reference sample.

Normalized relative expression gene1 sample1=(relative expression gene1 sample1/relative expression gene1 reference RNA)×100

Ct is the threshold cycle. The Ct is the cycle number at which the fluorescence generated within a reaction crosses the threshold line.

All experiments are normalized to a reference RNA, which is a comprehensive mix of RNA from various tissue sources (e.g., reference RNA #636538 from Clontech, Mountain View, Calif.). Identical reference RNA is included in each qRT-PCR run, allowing comparison of results between different experimental runs.

In one embodiment, the sample is a clinical sample. In another embodiment, the sample is used in a diagnostic assay. In some embodiments, the sample is obtained from tissue. Tissue biopsy is often used to obtain a representative piece of tissue. Alternatively, tumor cells can be obtained indirectly in the form of tissues or fluids that are known or thought to contain the cells of interest. Genes or gene products can be detected from tissue or from other body samples such as urine, sputum, serum or plasma. By screening such body samples, the progress of therapy can be monitored more easily by testing such body samples for target genes or gene products.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of disease and the test sample is later obtained when the disease has progressed.

In certain embodiments, a reference sample, reference cell, reference tissue, control sample, control cell, or control tissue is a combined multiple samples from one or more healthy individuals who are not the subject or individual. In certain embodiments, a reference sample, reference cell, reference tissue, control sample, control cell, or control tissue is a combined multiple samples from one or more individuals with a disease or disorder who are not the subject or individual. In certain embodiments, a reference sample, reference cell, reference tissue, control sample, control cell, or control tissue is pooled RNA samples from normal tissues or pooled plasma or serum samples from one or more individuals who are not the subject or individual. In certain embodiments, a reference sample, reference cell, reference tissue, control sample, control cell, or control tissue is pooled RNA samples from tissues or pooled plasma or serum samples from one or more individuals with a disease or disorder who are not the subject or individual.

In certain embodiments, a reference sample, reference cell, reference tissue, control sample, control cell, or control tissue is a sample cell line. In certain embodiments, a reference sample, reference cell, reference tissue, control sample, control cell, or control tissue is blood.

In some embodiments, the sample is a tissue sample from the individual. In some embodiments, the tissue sample is a blood or urine sample. In some embodiments, the tissue sample is a blood sample.

In some embodiments of any of the methods, the BTK inhibitor is a small molecule BTK inhibitor. In some embodiments, the small molecule BTK inhibitor is Compound (A) or a pharmaceutically acceptable salt thereof.

In some embodiments of any of the methods, the individual or patient according to any of the above embodiments may be a human.

In a further embodiment, provided herein are methods for treating SLE. In one embodiment, the method comprises administering to an individual having SLE an effective amount of a small molecule BTK inhibitor. In one such embodiment, the method further comprises administering to the individual an effective amount of at least one additional therapeutic agent, as described below. In some embodiments, the individual may be a human.

BTK inhibitors described herein can be used either alone or in combination with other agents in a therapy. For instance, the additional therapeutic may be an anti-inflammatory agent, an immunomodulatory agent, chemotherapeutic agent, an apoptosis-enhancer, a neurotropic factor, an agent for treating cardiovascular disease, an agent for treating liver disease, an anti-viral agent, an agent for treating blood disorders, an agent for treating diabetes, and an agent for treating immunodeficiency disorders. The second therapeutic agent may be an NSAID anti-inflammatory agent. The second therapeutic agent may be a chemotherapeutic agent. The second compound of the pharmaceutical combination formulation or dosing regimen preferably has complementary activities to the compound (I) such that they do not adversely affect each other.

In some embodiments, the additional therapeutic is selected from the group consisting of: corticosteroids (e.g., prednisone, prednisolone, methylprednisolone, and hydrocortisone); disease-modifying antihreumatic drugs (“DMARDs”, e g , immunosuppressive or anti-inflammatory agents); anti-malarial agents (e.g. hydroxychloroquine and chloroquine); immunosuppressive agents (e.g., cyclophosphamide, azathioprine, mycophenolate mofetil, methotrexate); anti-inflammatory agents (e.g., aspirin, NSAIDs (e.g., ibuprofen, naproxen, indomethacin, nabumetone, celecoxib)); anti-hypertensive agents (e.g., calcium channel blockers (e.g., amlodipine, nifedipine) and diuretics (e.g., furosemide)); statins (e.g., atorvastatin, fluvastatin, lovastatin, pitavastatin, pravastatin, rosuvastatin and simvastatin); anti-B-cell agents (e.g., anti-CD20 (e.g., rituximab), anti-CD22); anti-B-lymphocyte stimulator agents (“anti-BLyS”, e.g., belimumab, blisibimod); type-1 interferon receptor antagonist (e.g., anifrolumab); T-cell modulators (e.g., rigerimod); abatacept; anticoagulants (e.g., heparin, warfarin); and vitamin D supplements.

The combination therapy may be administered in a simultaneous or in a sequential regimen. When administered sequentially, the combination may be dosed in two or more administrations. The combined administration includes co-administration, using separate formulations or a single pharmaceutical formulation, and consecutive administration in either order, wherein preferably there is a time period while both (or all) active agents simultaneously exert their biological activities. Suitable dosages for any of the above co-administered agents are those presently used and may be lowered due to the combined action (synergy) of the additional therapeutic agents.

The combination therapy may be synergistic such that the effect achieved when the active ingredients used together is greater than the sum of the effects that results from using the compounds separately. A synergistic effect may be attained when the active ingredients are: (1) administered or delivered simultaneously; (2) administered in alternation or in parallel; or (3) by some other regimen. When delivered in alternation therapy, a synergistic effect may be attained when the compounds are administered or delivered sequentially. In general, during alternation therapy, an effective dosage of each active ingredient is administered sequentially, i.e., serially, whereas in combination therapy, effective dosages of two or more active ingredients are administered together.

In combination therapy, a kit may comprise (a) a first container with a dosage form composition of the present disclosure and, optionally, (b) a second container with a second pharmaceutical formulation contained therein for co-administration with the dosage form compositions of the present disclosure. In such aspects, the kit may comprise a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

A BTK inhibitor can be administered by any suitable means, including oral, parenteral, intrapulmonary, and intranasal, and, if desired for local treatment, intralesional administration. In preferred embodiments, the BTK inhibitor is administered orally.

Oral dosage forms comprising a BTK inhibitor include, but are not limited to, tablets or capsules comprising the BTK inhibitor or a pharmaceutically acceptable salt thereof, and one or more pharmaceutically acceptable excipients. In some embodiments, the tablet(s) or capsule(s) comprising the BTK inhibitor may be administered according to the methods provided herein, either once or twice daily. In certain embodiments provided herein, the oral dosage form is a tablet comprising Compound (A) or a pharmaceutically acceptable salt thereof, and one or more pharmaceutically acceptable excipients.

Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

BTK inhibitors described herein may be formulated, dosed, and administered in a fashion consistent with good medical practice. Factors for consideration in this context include the particular disease or disorder being treated, the particular mammal being treated, the clinical condition of the individual patient, the cause of the disease or disorder, the site of delivery of the agent, the method of administration, the scheduling of administration, and other factors known to medical practitioners. The BTTK inhibitor need not be, but is optionally formulated with one or more agents currently used to prevent or treat the disease or disorder in question. The effective amount of such other agents depends on the amount of the BTK inhibitor present in the formulation, the type of disease or disorder or treatment, and other factors discussed above. These are generally used in the same dosages and with administration routes as described herein, or about from 1 to 99% of the dosages described herein, or in any dosage and by any route that is empirically/clinically determined to be appropriate.

### III. Therapeutic Compositions Comprising BTK Inhibitors

Provided in the compositions, methods and kits herein are small molecule BTK inhibitors. Small molecule BTK inhibitors as provided herein are preferably organic molecules other than binding polypeptides or antibodies, and may be identified and chemically synthesized using known methodology. Binding organic small molecules are usually less than about 2000 daltons in size, alternatively less than about 1500, 750, 500, 250 or 200 daltons in size, wherein such organic small molecules that are capable of binding, preferably specifically, to BTK as described herein may be identified without undue experimentation using well known techniques. In this regard, it is noted that techniques for screening organic small molecule libraries for molecules that are capable of binding to a polypeptide target are well known in the art (see, e.g., PCT Publication Nos. WO 2000/00823 and WO 2000/39585). Binding organic small molecules may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

In some embodiments of any of the methods, the BTK inhibitor is selected from the group consisting of: ibrutinib, acalabrutinib, spebrutinib, BIIB068 (Biogen), BMS-986195 (Bristol-Myers Squibb), BMS-986142 (Bristol-Myers Squibb), BMS-935177 (Bristol-Myers Squibb), M2951 (Merck KGaA), PRN-1008 (Principia Biopharma), HM71224/LY3337641 (Hanmi/Lilly), ONO-4059/GS-4059 (Gilead/Ono), AC0058 (ACEA Biosciences), AC0025 (ACEA Biosciences), ABBV-599 (AbbVie), ABBV-105 (AbbVie), PF-303 (Pfizer), BI-BTK1 (Boehringer Ingelheim), CC90008 (Celgene), AS550 (Cama Biosciences), ARQ 531 (Arqule), AEG42766 (Aegera Therapeutics), BGB-3111 (Beigene), RN486 (Simcere Pharma), HCI-1401 (LSK BioPharma/Hustman Cancer Inst.), KBP-7536 (KBP Bioscience), RDX002 (RedX Biopharma), SNS-062 (Sunesis), TAS5315 (Taiho Pharma), TAX-020 (Takeda), WX486/WXFL-10230486 (WuXi AppTec/Humanwell), and X-022 (X-Rx Discovery).

In some embodiments, the BTK inhibitor is Compound (A) or a pharmaceutically acceptable salt thereof. Pharmaceutically acceptable salts of a BTK inhibitor provided herein may be used in the methods herein. As used herein, the term “pharmaceutically acceptable salt” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. As used herein the term “prodrug” refers to those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Prodrugs of the invention include compounds wherein an amino acid residue, or a polypeptide chain of two or more (e.g., two, three or four) amino acid residues, is covalently joined through an amide or ester bond to a free amino, hydroxy or carboxylic acid group of a compound of the present invention. The amino acid residues include but are not limited to the 20 naturally occurring amino acids commonly designated by three letter symbols and also includes phosphoserine, phosphothreonine, phosphotyrosine, 4-hydroxyproline, hydroxylysine, demosine, isodemosine, gamma-carboxyglutamate, hippuric acid, octahydroindole-2-carboxylic acid, statine, 1,2,3,4-tetrahydroisoquinoline-3-carboxylic acid, penicillamine, ornithine, 3-methylhistidine, norvaline, beta-alanine, gamma-aminobutyric acid, citrulline, homocysteine, homoserine, methyl-alanine, para-benzoylphenylalanine, phenylglycine, propargylglycine, sarcosine, methionine sulfone and tert-butylglycine.

Additional types of prodrugs are also encompassed. For instance, a free carboxyl group of a compound of the invention can be derivatized as an amide or alkyl ester. As another example, compounds of this invention comprising free hydroxy groups can be derivatized as prodrugs by converting the hydroxy group into a group such as, but not limited to, a phosphate ester, hemisuccinate, dimethylaminoacetate, or phosphoryloxymethyloxycarbonyl group, as outlined in Fleisher, D. et al., (1996) Improved oral drug delivery: solubility limitations overcome by the use of prodrugs Advanced Drug Delivery Reviews, 19:115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers, wherein the acyl group can be an alkyl ester optionally substituted with groups including, but not limited to, ether, amine and carboxylic acid functionalities, or where the acyl group is an amino acid ester as described above, are also encompassed. Prodrugs of this type are described in J. Med. Chem., (1996), 39:10. More specific examples include replacement of the hydrogen atom of the alcohol group with a group such as (C₁₋₆)alkanoyloxymethyl, 1-((C₁₋₆)alkanoyloxy)ethyl, 1-methyl-1-((C₁₋₆)alkanoyloxy)ethyl, (C₁₋₆)alkoxycarbonyloxymethyl, N-(C₁₋₆)alkoxycarbonylaminomethyl, succinoyl, (C₁₋₆)alkanoyl, alpha-amino(C₁₋₄)alkanoyl, arylacyl and alpha-aminoacyl, or alpha-aminoacyl-alpha-aminoacyl, where each alpha-aminoacyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

For additional examples of prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8:1-38 (1992); d) H. Bundgaard, et al., Journal of Pharmaceutical Sciences, 77:285 (1988); and e) N. Kakeya, et al., Chem. Pharm. Bull., 32:692 (1984), each of which is specifically incorporated herein by reference.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention can exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers, regioisomers and individual isomers (e.g., separate enantiomers) are all intended to be encompassed within the scope of the present invention.

### IV. Pharmaceutical Formulations

Pharmaceutical formulations of a BTK inhibitor are provided in the methods and kits herein.

In some embodiments of any of the methods, the BTK inhibitor (e.g., Compound (A) or a pharmaceutically acceptable salt thereof) is administered at a dosage of about 0.1 mg/kg/day to about 100 mg/kg/day, from about 0.5 mg/kg/day to about 20 mg/kg/day, or from about 1 mg/kg/day to about 10 mg/kg/day on the basis of patient body weight. In some embodiments, Compound (A) or a pharmaceutically acceptable salt thereof, is administered as a tablet at a dosage of about 10 to 800 mg. In some embodiments, Compound (A) is administered as a free base in a tablet at a dosage of about 25 to 300 mg. In some embodiments, the tablet comprises 25 to 300 mg of Compound (A) as a free base, and fumaric acid, wherein the weight ratio of Compound (A) to fumaric acid is from about 1:5 to about 3:1; or from about 1:2 to about 2:1; or from about 1:1.5 to about 1.5:1. In some embodiments, the tablet comprises 25 to 300 mg of Compound (A) as a free base, and fumaric acid, and wherein the fumaric acid content is from about 5 wt. % to about 50 wt. %, from about 5 wt. % to about 40 wt. %, from about 5 wt. % to about 30 wt. %, from about 10 wt. % to about 30 wt. %, from about 20 wt. % to about 25 wt. %, from about 5 wt. % to about 15 wt. %, or from about 10 wt. % to about 15 wt. %. In some of the above embodiments, the tablet weight is about 100 mg, about 200 mg, about 300 mg, about 400 mg, about 500 mg, about 600 mg, about 700 mg, about 800 mg, about 900 mg, or about 1000 mg. In some embodiments, the tablet further comprises at least one pharmaceutically acceptable excipient selected from fillers, binders, disintegrants, lubricants and glidants. In some embodiments, the tablet comprises lactose and microcrystalline cellulose.

The tablet compositions of the present disclosure may further suitably comprise one or more pharmaceutically acceptable excipients selected from, but not limited to fillers (diluents), disintegrants, binders, glidants, and lubricants. A filler (or diluent) may be used to increase the bulk volume of the powdered drug making up the tablet. A disintegrant may be used to encourage the tablet to break down into small fragments, ideally individual drug particles, when it is ingested and thereby promote the rapid dissolution and absorption of drug. A binder may be used to ensure that granules and tablets can be formed with the required mechanical strength and hold a tablet together after it has been compressed, preventing it from breaking down into its component powders during packaging, shipping and routine handling A glidant may be used to improve the flowability of the powder making up the tablet during production. A lubricant may be used to ensure that the tableting powder does not adhere to the equipment used to press the tablet during manufacture, to improve the flow of the powder during mixing and pressing, and to minimize friction and breakage as the finished tablets are ejected from the equipment.

Fillers and binders may include calcium hydrogenphosphate, microcrystalline cellulose (Avicel®), lactose, or any other suitable bulking agent. Examples of suitable fillers include microcrystalline cellulose, such as Avicel PH 101, Avicel PH102, Avicel PH 200, Avicel PH 105, Avicel DG, Ceolus KG 802, Ceolus KG 1000, SMCCSO and Vivapur 200; lactose monohydrate, such as Lactose FastFlo; microcrystalline cellulose co-processed with other excipients, such as microcrystalline cellulose coprocessed with lactose mono hydrate (MicroceLac 100) and microcrystalline cellulose co-processed with colloidal silicon dioxide (SMCCSO, Prosolv 50 and Prosolv HD 90); mixtures of isomaltulose derivatives such as galenlQ; and other suitable fillers and combinations thereof. The filler may be present as an intra-granular component and/or as an extra-granular component. In some particular aspects, the tablet compositions of the present disclosure comprise lactose and microcrystalline cellulose.

Disintegrants may be included in the disclosed formulations to promote separation of the granules within the compact from one another and to maintain separation of the liberated granules from one another. Distintegrants may be present as an intra-granular component and/or as an extra-granular component. Disintegrants may include any suitable disintegrant such as, for example, crosslinked polymers such as cross-linked polyvinyl pyrrolidone and cross-linked sodium carboxymethylcellulose or croscarmellose sodium. In some particular aspects, the disintegrant is croscarmellose sodium. The disintegrant content is suitably about 1 wt. %, about 1.5 wt. %, about 2 wt. %, about 2.5 wt. %, about 3 wt. %, about 3.5 wt. %, about 4 wt. %, about 4.5 wt. %, or about 5 wt. %, and ranges thereof, such as from about 1 wt. % to about 5 wt. %, or from about 2 wt. % to about 4 wt. %.

Glidants may include, for example, colloidal silicon dioxide, including highly dispersed silica (Aerosil®), or any other suitable glidant such as animal or vegetable fats or waxes. In some particular aspects, the glidant is fumed silica. The glidant content is suitably about 0.1 wt. %, about 0.5 wt. %, about 1 wt. %, about 1.5 wt. %, about 2 wt. %, about 2.5 wt. % or about 3 wt. %, and ranges thereof, such as from about 0.1 wt. % to about 3 wt. %, from about 0.5 wt. % to about 2 wt. %, from about 0.5 wt. % to about 1.5 wt. %.

Lubricants may be used in compacting granules in the pharmaceutical composition. Lubricants may include, for example, polyethylene glycol (e.g., having a molecular weight of from about 1000 to about 6000), magnesium and calcium stearates, sodium stearyl fumarate, talc, or any other suitable lubricant. In some particular aspects, the lubricant is magnesium stearate and/or sodium stearyl fumarate. The lubricant may be present as an intra-granular component and/or as an extra-granular component. The lubricant content is suitably about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, or about 5 wt. %, and ranges thereof, such as from about 0.5 wt. % to about 5 wt. %, from about 1 wt. % to about 4 wt. %, from about 1 wt. % to about 3 wt. %, or from about 1 wt. % to about 2 wt. %.

A coating, such as a film coating, may be applied to the tablets of the present disclosure. A film coat may be used to, for example, contribute to the ease with which the tablet can be swallowed. A film coat may also be employed to improve taste and appearance. If desired, the film coat may be an enteric coat. The film coat may comprise a polymeric film-forming material such as hydroxypropyl methylcellulose, hydroxypropyl cellulose, acrylate or methacrylate copolymers, and polyvinyl alcohol-polyethylene glycol graft copolymers such as Opadry and Kollicoat IR. In addition to a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as an anti-adhesive. The film coat typically accounts for less than about 5% by weight of the dosage form.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the BTK inhibitor, which matrices are in the form of shaped articles, e.g., films, or microcapsules.

The formulations to be used for in vivo administration are generally sterile. Sterility may be readily accomplished, e.g., by filtration through sterile filtration membranes.

### V. Articles of Manufacture

In another embodiment, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a BTK inhibitor described herein. The label or package insert indicates that the composition is used for treating the condition of choice. Moreover, the article of manufacture may comprise (a) a first container with a composition contained therein, wherein the composition comprises a BTK inhibitor; and (b) a second container with a composition contained therein, wherein the composition comprises a further cytotoxic or otherwise therapeutic agent.

In some embodiments, the article of manufacture comprises a container, a label on said container, and a composition contained within said container; wherein the composition includes one or more reagents (e.g., primary antibodies (e.g., B-9 Santa Cruz Biotechnology antibody) that bind to one or more biomarkers or probes and/or primers to one or more of the biomarkers described herein), the label on the container indicating that the composition can be used to evaluate the presence of one or more biomarkers in a sample, and instructions for using the reagents for evaluating the presence of one or more biomarkers in a sample. The article of manufacture can further comprise a set of instructions and materials for preparing the sample and utilizing the reagents. In some embodiments, the article of manufacture may include reagents such as both a primary and secondary antibody, wherein the secondary antibody is conjugated to a label, e.g., an enzymatic label. In some embodiments, the article of manufacture one or more probes and/or primers to one or more of the biomarkers described herein.

The article of manufacture in this embodiment may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Other optional components in the article of manufacture include one or more buffers (e.g., block buffer, wash buffer, substrate buffer, etc.), other reagents such as substrate (e.g., chromogen) which is chemically altered by an enzymatic label, epitope retrieval solution, control samples (positive and/or negative controls), control slide(s) etc.

### EXAMPLES

The following are examples of methods and compositions. It is understood that various other embodiments may be practiced, given the general description provided above.

Blood Sample Analysis: 3-gene plasmablast signature expression assay. Blood was collected in PAXgene RNA tubes (PreAnalytiX); total RNA was extracted using commercially available kits according to manufacturer instructions (Qiagen).

Biomarkers: IgJ, TXNDC5, MZB1. Reference gene: TMEM55B

Expression of candidate biomarker genes in blood samples was assessed by Human Genome U133 Plus 2.0 arrays (Affymetrix Inc., Santa Clara, Calif.). Microarray hybridization was performed by Asuragen Inc. (Austin, Tex.). Raw CEL file data was summarized and normalized using Robust Multi-array Averaging (RMA) and analyzed using R and Bioconductor.

Alternatively, expression of candidate biomarker genes in blood samples was quantified by Fluidigm qPCR assays. 3-gene score was calculated from the mean of IgJ, TXNDC5 and MZB1, and normalized using reference gene TMEM55B. Blood samples were assessed using this assay developed on the Cobas 4800 platform (Roche Molecular Systems).

### Example 1

### Characterization of Plasmablast Transcriptome

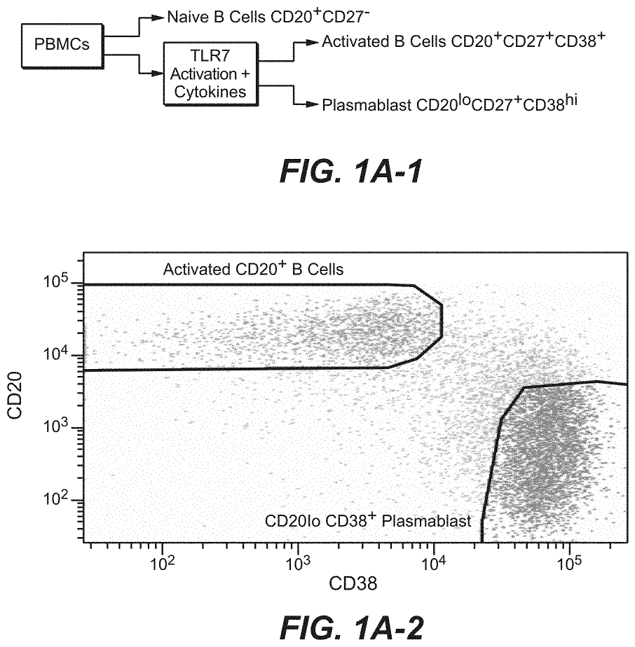

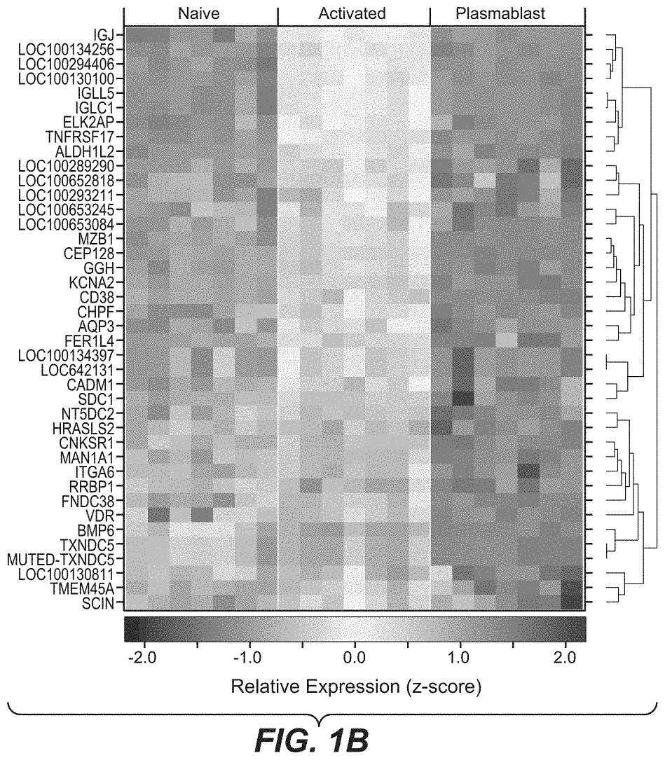

Transcriptional profiling of in vitro differentiated CD20^{lo}CD38⁺ plasmablasts, CD20⁺CD27⁺ activated B cells and CD20⁺CD27⁻ naïve B cells was performed to identify genes with strong differential expression between B cell subsets (FIG. 1A-1). 86 genes were identified that were expressed >10-fold higher in plasmablasts than either activated B cells or naïve B cells, at a false discovery rate (FDR) of 0.001. Further refinement of these data was performed to include only genes with >5 nRPKM in plasmablasts, yielding a total of 40 genes. Many of these genes included heavy and light chain segments, as well as genes involved in the biosynthesis of immunoglobulin proteins. Biomarker candidates were selected that were not part of the immunoglobulin locus, so these were removed from the list of candidate genes (FIG. 1B).

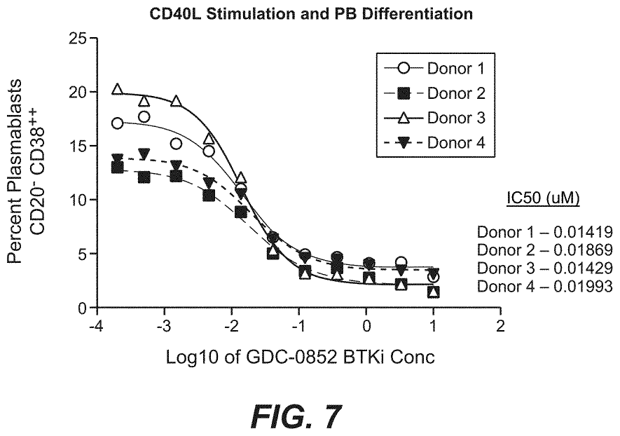

It was confirmed that plasmablast differentiation was regulated by Bruton's Tyrosine Kinase (BTK) activity by performing an in vitro plasmablasts differentiation assay where human memory B cells were put into differentiating conditions, and were subsequently measured after 5 days using flow cytometry quantification for CD20^{lo}CD38⁺⁺ plasmablasts. Use of a specific and potent inhibitor of BTK kinase activity, GDC-0852, inhibited CD40L induced plasmablast differentiation in a dose-dependent manner (FIG. 7). GDC-0852 is (S)-2-(5-Fluoro-2-(hydroxymethyl)-3-(1-methyl-5-(5-(2-methyl-4-(oxetan-3-y-1)piperazin-1-yl)pyridin-2-ylamino)-6-oxo-1,6-dihydropyridin-3-yl)-phenyl)-3,4,6,7,8,9-hexahydropyrido[3,4-b]indolizin-1(2H)-one, the structure of which is shown below:

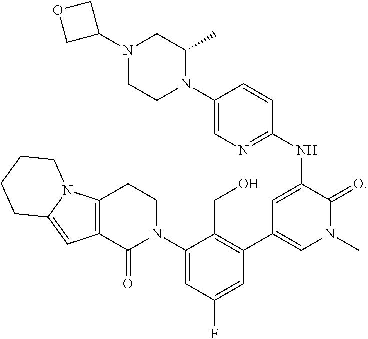 [SMILES: C[C@H]1CN(C2COC2)CCN1c1ccc(Nc2cc(-c3cc(F)cc(N4CCc5c(cc6n5CCCC6)C4=O)c3CO)cn(C)c2=O)nc1]

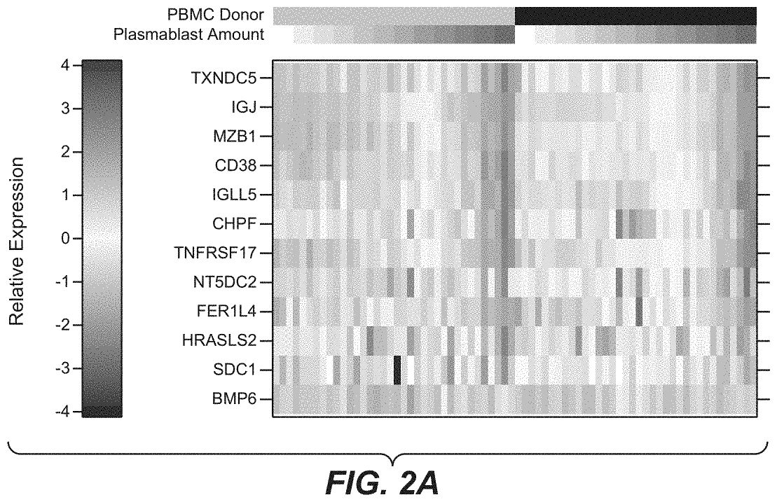

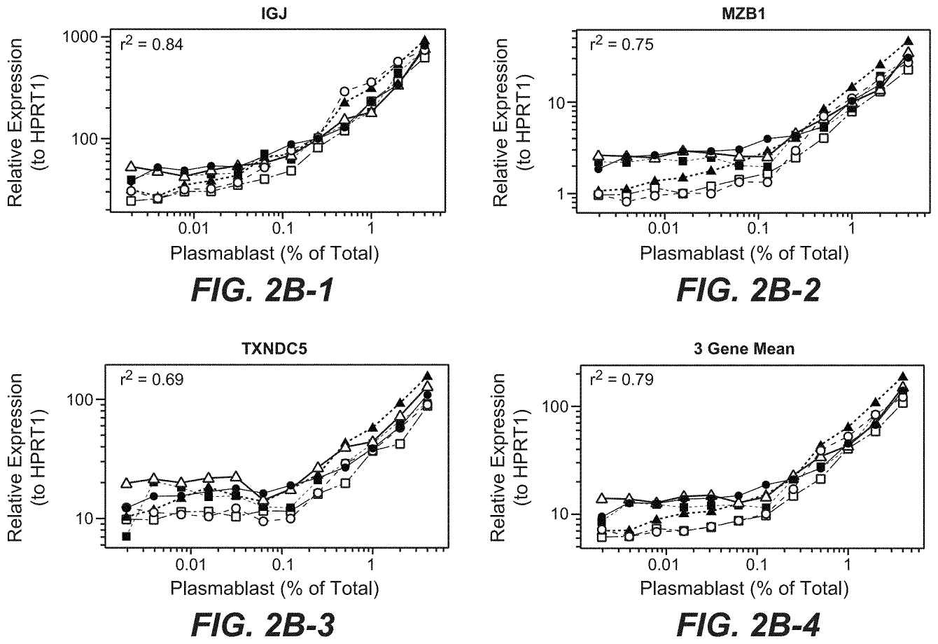

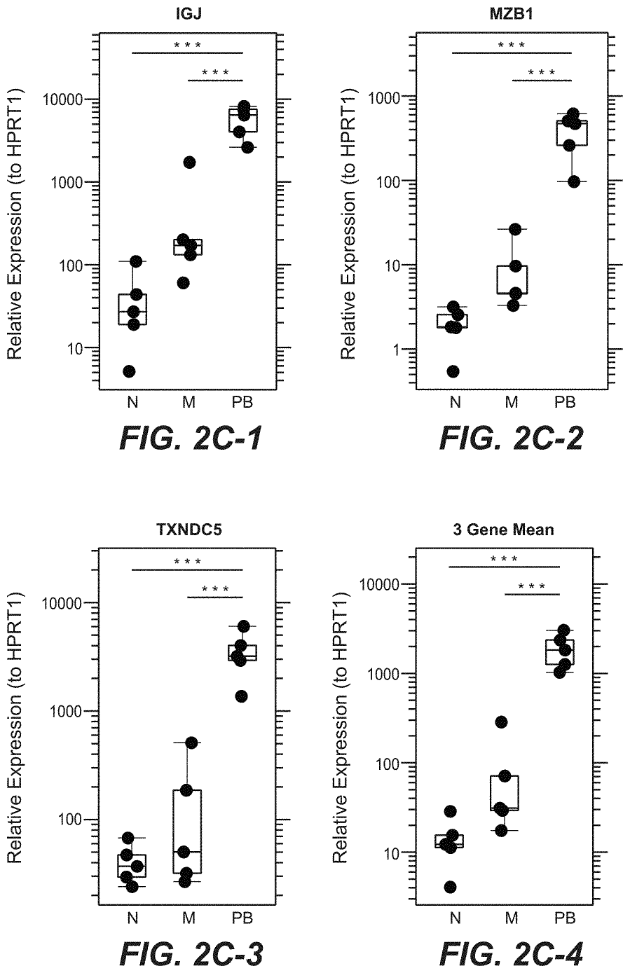

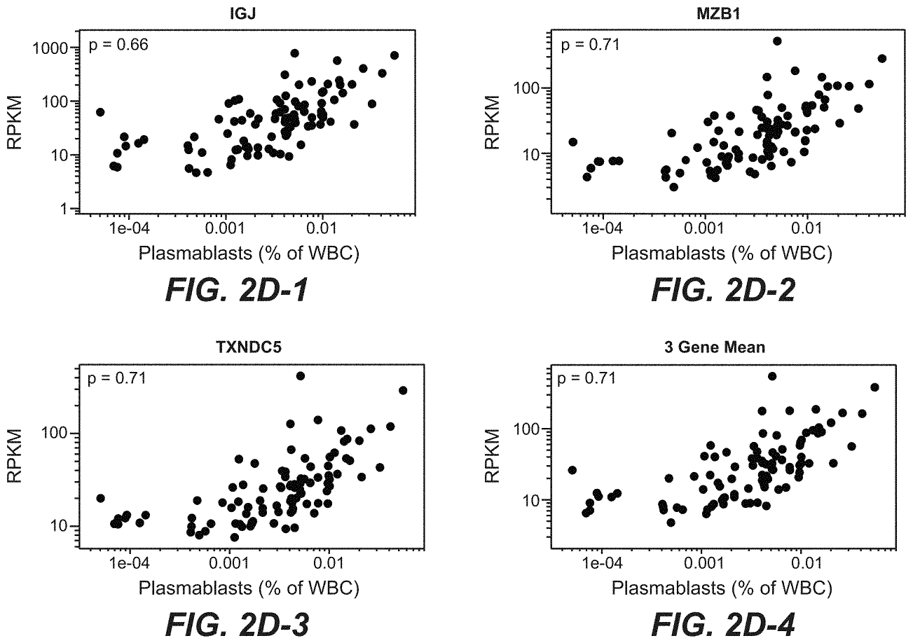

To ensure that chosen biomarker candidates could accurately determine the fraction of plasmablasts in a sample with high sensitivity and specificity, serial 2-fold dilution was performed, from 40,000 to 39 cells, of in vitro differentiated plasmablasts derived from 3 donors into 1,000,000 PBMCs derived from two separate donors. Expression levels of candidate plasmablast marker genes were evaluated using Fluidigm, reported as ΔCt relative to a housekeeping gene, HPRT1. Linear regression was used to model the ΔCt of the candidate genes predicted by the log₁₀ plasmablast frequency, with plasmablast donor and PBMC donor as covariates. Most of our candidate genes showed a strong association with plasmablast frequency, and minimal differences between plasmablast donors. Three genes, IGJ, MZB1 and TXNDC5, performed particularly well, showing r² of 0.84, 0.75 and 0.69, respectively (FIG. 2B-1, 2B-2, 2B-3, 2B-4). Taking the mean of the three genes as a signature score yielded a signature score with an r² of 0.79.

To validate the gene signature in in vivo differentiated plasmablasts, their expression in plasmablasts directly sorted from five healthy donors who had been vaccinated with influenza vaccine one week prior were measured. All three of the candidate biomarker genes were more highly expressed in plasmablasts relative to both naive and memory B cells (FIG. 2C-1, 2C-2, 2C-3, 2C-4).

### Example 2

### Plasmablast Marker Genes Correlate with Plasmablast Frequency In Vivo

The frequency of plasmablasts in whole blood was measured using flow cytometry in a cohort of lupus patients from the ROSE phase II clinical trial [3], which had paired RNA-sequencing data. The selected plasmablast signature genes were shown to display a high correlation with the frequency of IgD⁻CD19⁺CD27⁺⁺CD38⁺⁺ plasmablasts (FIG. 2D-1, 2D-2, 2D-3, 2D-4). IGJ, MZB1 and TXNDC5 show among the highest correlation coefficients with the plasmablast content, with Spearman correlation coefficients of 0.66, 0.71 and 0.71, respectively. Taking the mean of the three signature genes as a signature shows a strong correlation with plasmablast frequency (Spearman p=0.71). These results demonstrate that the relative abundance of plasmablasts can be measured in whole blood samples using the selected three gene signature.

### Example 3

### Plasmablast Signature Correlates with Increased Disease Activity in SLE

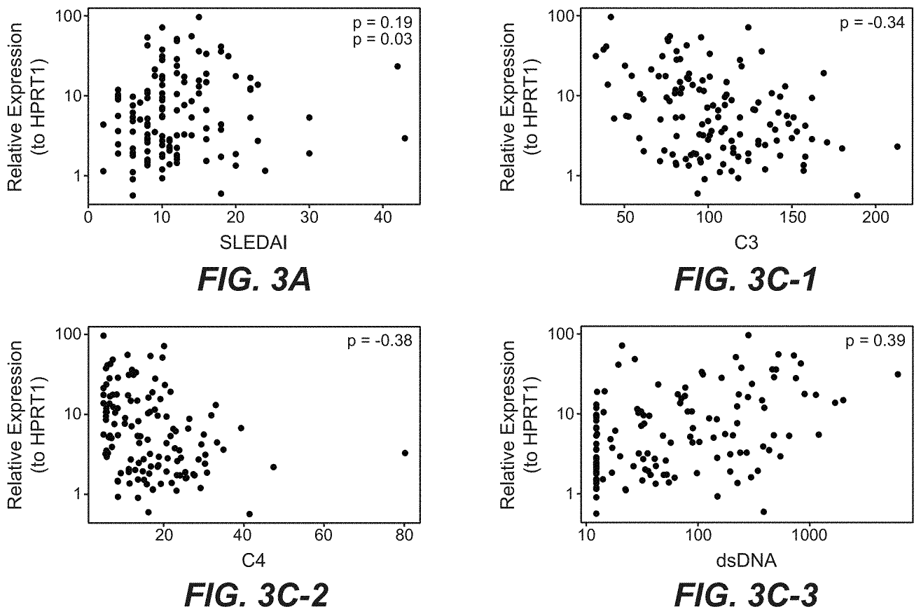

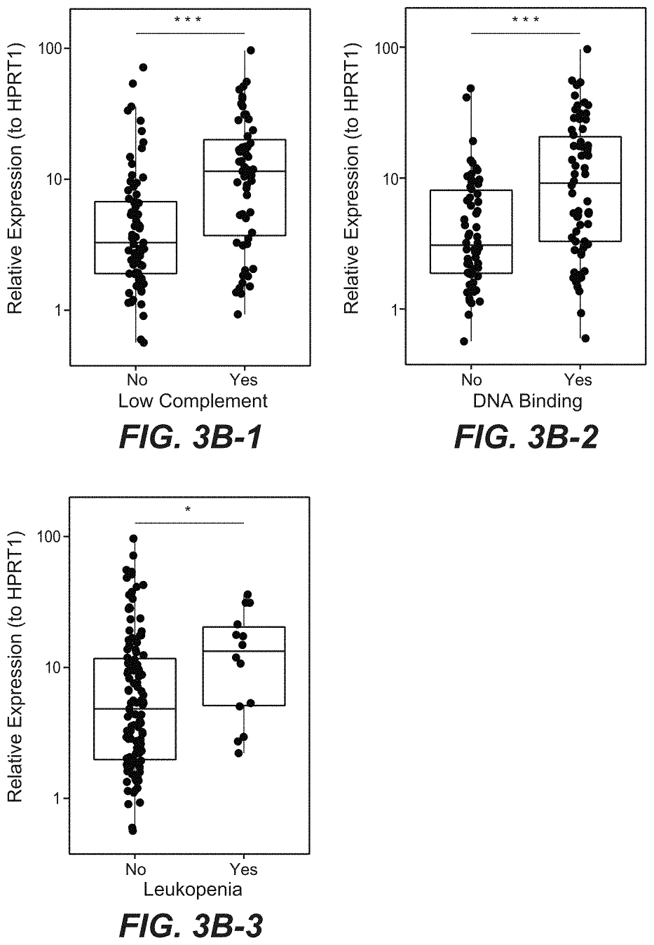

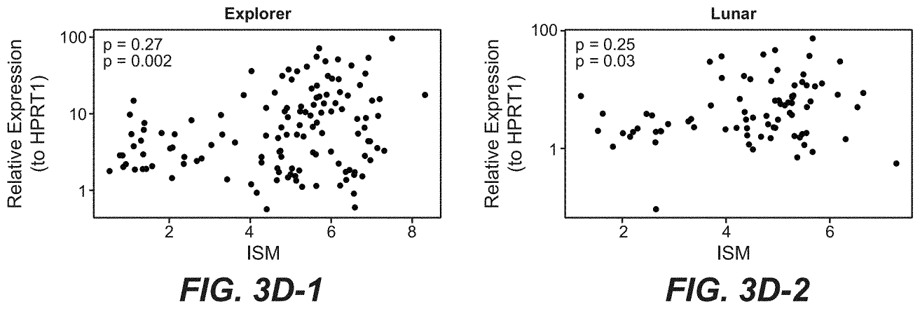

Previous work has demonstrated a strong correlation between plasmablast frequency and disease severity as measured by the Safety of Estrogen in Lupus Erythematosus (SELENA)—Systemic Lupus Erythematosus Disease Activity Index (SLEDAI) score. This correlation appears to be driven by differences in patients with quiescent disease and active disease. Looking within a cohort of moderate to severe extra-renal lupus from the EXPLORER phase II clinical trial [4], it was found that the plasmablast signature showed a low, but significant correlation with disease activity (Spearman p=0.19, p=0.03, FIG. 3A). Looking more closely into the SLEDAI components that drive this correlation, three subscores in particular were found to be associated with higher plasmablast abundance: DNA binding, low complement and lymphopenia (FIG. 3B-1, 3B-2, 3B-3).

A moderate negative correlation was discovered between plasmablast abundance and serum concentrations of complement components C3 and C4 in multiple lupus patient cohorts (Spearman's p=−0.34, −0.38, respectively, FIG. 3C). A moderate correlation between plasmablast content and the titer of anti-double stranded DNA antibodies in these cohorts was also observed (Spearman's p=0.39, FIG. 3C-1, 3C-2, 3C-3).

The majority of lupus patients show a transcriptional signature of interferon activity [2,5]. The plasmablast signature showed a moderate correlation with interferon activity measured using a three-gene signature (FIG. 3D-1 and 3D-2; [5]). The correlation appeared to be driven by elevation of plasmablast signature expression in a subset of patients with high levels of interferon activity, with most low interferon signature patients having low expression of plasmablast genes, while patients with high interferon activity showed a mix of low and high levels of plasmablast gene expression. However, the plasmablast signature correlates with disease severity and serological activity independently of the interferon signature; using backward model selection, with Akaike information criterion as the metric, the plasmablast and interferon signatures were predictive of serum complement levels and the plasmablast signature alone was predictive of anti-dsDNA antibody titer and SLEDAI.

No association was seen between plasmablast signature scores and the British Isles Lupus Assessment Group (BILAG) activity index, either as a composite score, or for individual disease domains. These data support a role of plasmablasts in serological disease activity driven by autoantibodies, lymphopenia and hypocomplementemia.

### Example 4

### Rituximab Treatment Reduces Plasmablast Signature

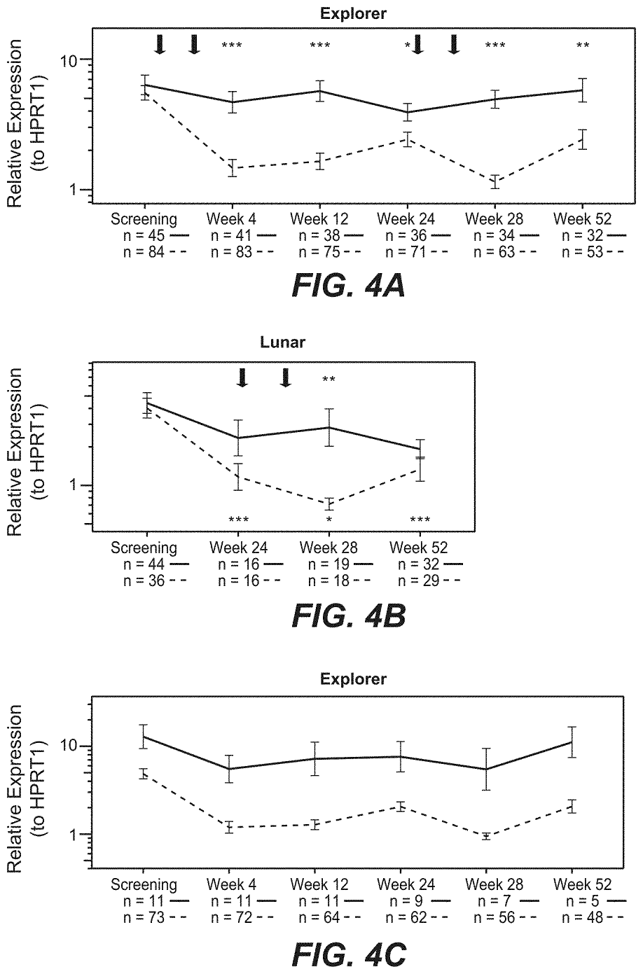

Plasmablast signature values were collected from two cohorts of moderate to severe lupus patients from phase II clinical trials assessing the safety and efficacy of rituximab in SLE. The cohorts were patients with either lupus nephritis (LUNAR) or extra-renal lupus (EXPLORER) [4,6]. Mixed effects modeling of plasmablast signature values over the course of treatment, incorporating covariates for age, race, concomitant medications, interferon activity, SLEDAI, visit, treatment arm and their interaction, with patient modeled as a random effect, identified a profound decrease in plasmablast signature specifically within the rituximab treated patients (FIG. 4A, 4B). This effect was most apparent in the time points two weeks after receiving rituximab infusion, with diminishing effect over time. In the EXPLORER trial, a maximum decrease of 3.48 fold was observed at week 28, two weeks following the fourth infusion of rituximab (p=2×10⁻¹¹). Likewise, in the LUNAR trial, the lowest levels of plasmablast signature expression were observed at the week 28 time point, with a 3.31 fold reduction (p=0.0011).

In the EXPLORER trial, patients were monitored for presence of antibodies directed against mouse-human chimeric antibodies (HACA). While most rituximab-treated patients showed a decrease in plasmablast signature, it was observed that patients that went on to develop anti-drug antibodies showed limited to no decrease in plasmablast signature after rituximab treatment (FIG. 4C). Using the same linear mixed effects model, as above, a 2.8-fold higher expression of plasmablast marker genes was found in patients that went on to develop HACA (p=0.0007).

### Example 5

### Lupus Standard of Care Treatment Alters Plasmablast Signature

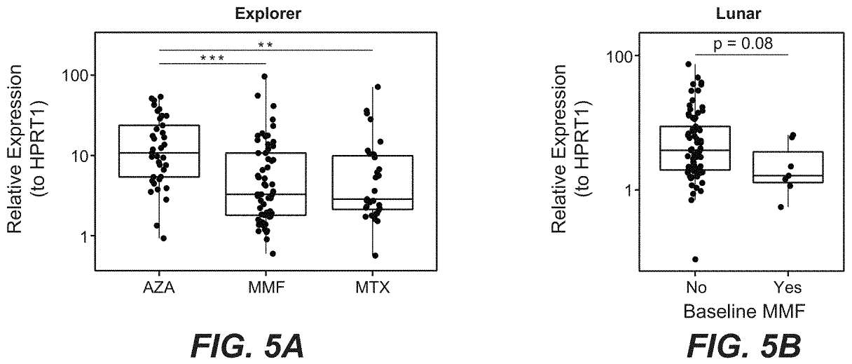

Previous studies identified an association between different immunosuppressive treatments and reduced expression of plasmablast-associated genes [7]. Looking at the screening samples from the EXPLORER clinical trial, much lower expression of the plasmablast signature was found in patients treated with mycophenolate or methotrexate compared to patients treated with azathioprine (FIG. 5A). A trend toward lower expression of plasmablast signature genes in patients from the LUNAR trial was observed in patients that were on mycophenolate treatment at baseline (FIG. 5B), though relatively few patients were in the MMF treatment pool.

### Example 6

### Patient Race/Ethnicity Affects Plasmablast Signature Levels

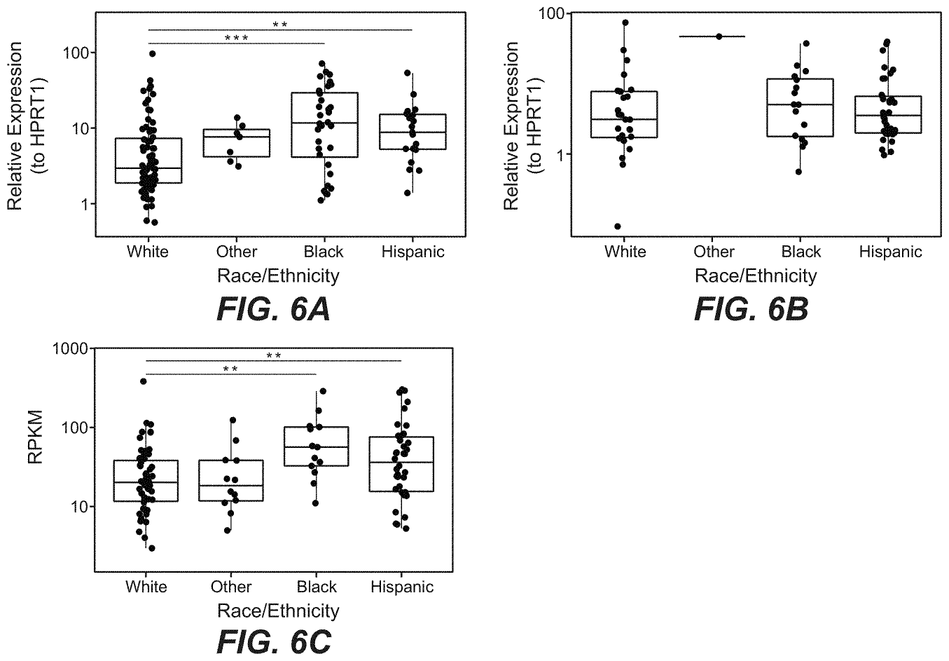

Biomarker levels are frequently different between patient populations. Data analysis revealed significantly lower levels of plasmablast signature in patients of European ancestry relative to patients of African or Hispanic ancestry in both EXPLORER and ROSE clinical trial populations. This held true when accounting for interferon activity, age and disease severity (FIG. 6A, 6B, 6C).

### Example 7

### Effect of BTK Inhibition on Plasmablast Differentiation

Plasmablast Differentiation: Memory B cells were isolated from Healthy donor PBMC (Miltenyi Memory B cell isolation kit). For the Plasmablast differentiation, 1.5×10\hat{ }5/ml memory B cells were then cultured in the presence of a cocktail of cytokines, IL-2(20 U/ml), IL-10 (50 ng/ml), IL-15(10 ng/ml), IL-6 (50 ng/ml), IFNa (10 ng/ml) and stimulated with either ODN2006 (TLR-9 ligand) 5 ug/ml, or CD40L (3 ug/ml) for 5 days. The plasmablast differentiation was carried out in the presence of vehicle alone (DMSO) and GDC-0852 at various concentrations, using a 3 fold dose titration of inhibitor starting at 10 uM. Flow cytometry was performed to enumerate (CD20⁻CD38⁻⁺) plasmablast percentages and assess the inhibition by GDC-0852.

RNA Preparation: RNA was extracted from the cells in culture at 5 days from the DMSO and GDC-0852 (370 nM) treated cells (n=4). Cells were disrupting in RLT buffer using the Qiashredder (Qiagen, Valencia, Calif.) and then RNA was extracted using the RNeasy mini kit (Qiagen) including the on-column DNase digestion. The concentration of total RNA and integrity of RNA samples was determined using the NanoDrop 8000 (Thermo Scientific). The isolated RNA was used for the Fluidigm quantitative RT-PCR analyses.

QT PCR: cDNA synthesis was performed on 100 ng total-RNA using an iScript cDNA synthesis kit (Biorad, Hercules, Calif.). Gene-specific pre-amplification was performed (Applied Biosystems) for 3 genes (IgJ, MZB1, TXNDC5 including housekeeping genes TMEM55B. RT-PCR was performed using the BioMark 48.48 Dynamic Arrays (Fluidigm Corporation) using the manufacturer's protocol. Data were collected using the BioMark Data Collection Software and CT values were obtained using the BioMark RT-PCR Analysis Software (V.2.1.1, Fluidigm). The relative abundance (dCt) to HPRT1 was calculated: 2 log−(average Ct gene−average Ct HPRT1). For statistical analyses, values below the lower limit of detection were set to be 1 Ct lower than the lowest recorded value.

Statistical analyses were performed using either <> or custom scripts written in the R programming language. To identify differences in gene expression, we fit a linear mixed effects model to the log 2-transformed relative transcript abundance, with treatment as a fixed effect, and donor as a random effect. To compare percentages of plasmablasts between DMSO and compound treated samples, we used a Wilcoxon rank sum test. IC50 values for BTK inhibition was calculated using the GraphPad Prism software. Human recombinant interleukin (IL)-2, and interferon-α (IFN-α) were purchased from R&D systems (Minneapolis, Minn.) and IL-10, IL-6 and IL-15 from Peprotech (Rocky Hill, N.J.). CpG (ODN2006) was purchased from Invivogen (San Deigo, Calif.) and CD40L was purchased from R&D systems (Minneapolis, Minn.).

Results: The differentiation of B cells into plasmablast can occur through multiple activation stimuli and involves distinct molecular changes. Activation of B cells through CD40, and/or Toll like receptor (TLR) results in differentiation of CD20⁺ CD27⁺⁺ memory B cells to CD20⁻CD38⁺⁺ plasmablasts. We evaluated the effect of the BTK inhibitor GDC-0852 in the plasmablast differentiation process, either by a T cell-mediated response using CD40L stimulation or a T cell-independent response using the Toll-like receptor ligand, CpG.

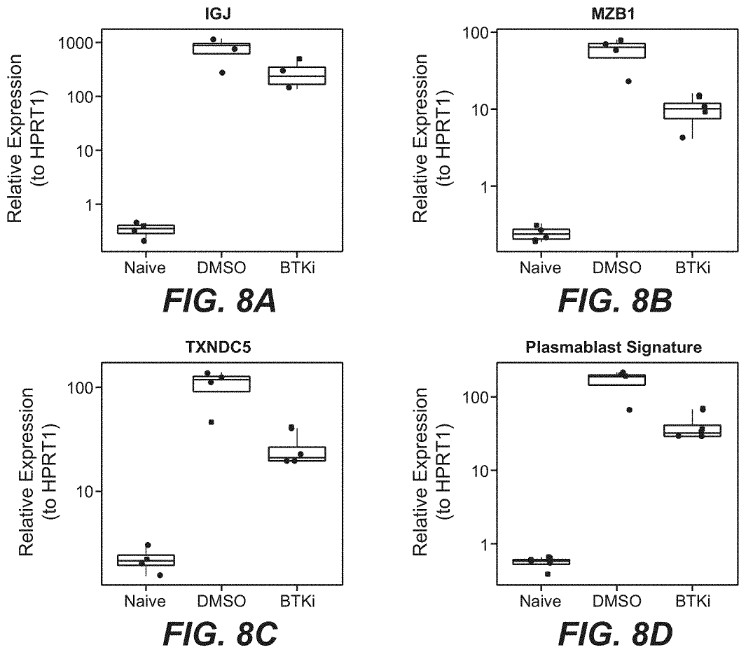

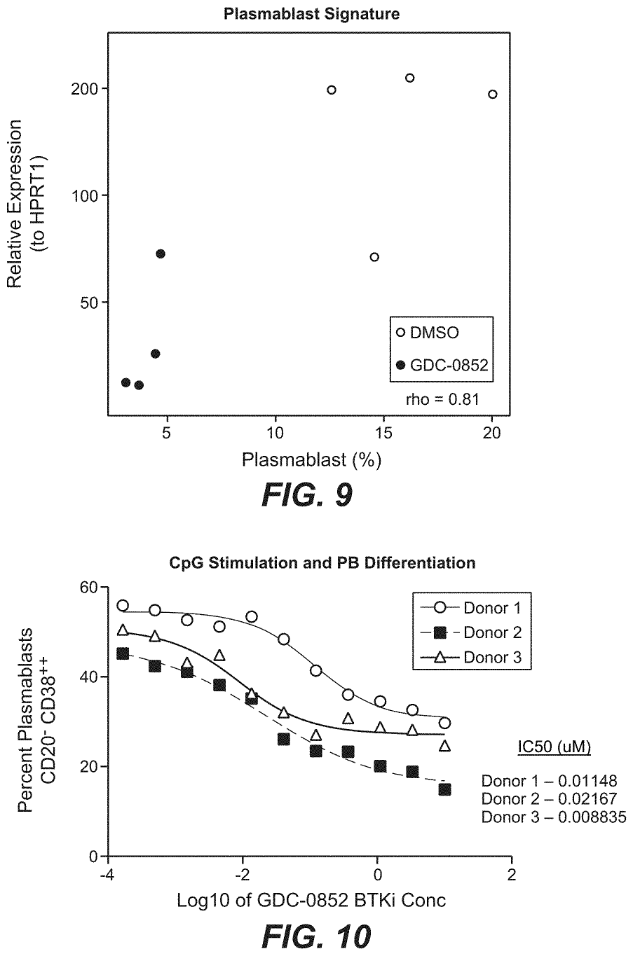

GDC-0852 inhibited CD40L induced Plasmablast differentiation in a dose dependent manner, on day 5, with an IC50 potency of 20.0 nM (+/−0.002) (FIG. 7). Gene expression analysis of the DMSO and GDC-0852 treated cells showed a significant decrease in the plasmablast signature genes IgJ (p=0.011), MZB1 (p=0.0023), TXNDC5 (p=0.0032) and the composite plasmablast 3 gene signature (p=0.0026), while naive B cells showed very low levels of expression of the signature genes (p<1×10⁻⁶) (FIG. 8). Comparing the gene expression values to plasmablast abundance, we see a strong correlation (Spearman rho=0.81) between the 3 gene signature and percent of plasmablasts (FIG. 9). CpG-mediated plasmablast differentiation (n=3), was also inhibited by GDC-0852, with an IC50 potency of 48 nM (+/−57) (FIG. 10).

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

Although the foregoing invention has been described in some detail by way of illustration and example for purposes of clarity of understanding, the descriptions and examples should not be construed as limiting the scope. The disclosures of all patent and scientific literature cited herein are expressly incorporated in their entirety by reference.

### REFERENCES

1. Arce E, Jackson D G, Gill M A, Bennett L B, Banchereau J, Pascual V. Increased frequency of pre-germinal center B cells and plasma cell precursors in the blood of children with systemic lupus erythematosus. J Immunol. 2001; 167: 2361-2369. doi:10.4049/jimmuno1.167.4.2361
2. Bennett L, Palucka a K, Arce E, Cantrell V, Borvak J, Banchereau J, et al. Interferon and granulopoiesis signatures in systemic lupus erythematosus blood. J Exp Med. 2003; 197: 711-723. doi:10.1084/jem.20021553
3. Kalunian K C, Merrill J T, Maciuca R, McBride J M, Townsend M J, Wei X, et al. A Phase II study of the efficacy and safety of rontalizumab (rhuMAb interferon-α) in patients with systemic lupus erythematosus (ROSE). Ann Rheum Dis. 2016; 75: 196-202. doi:10.1136/annrheumdis-2014-206090
4. Merrill J T, Neuwelt C M, Wallace D J, Shanahan J C, Latinis K M, Oates J C, et al. Efficacy and safety of rituximab in moderately-to-severely active systemic lupus erythematosus: The randomized, double-blind, phase II/III systemic lupus erythematosus evaluation of rituximab trial. Arthritis Rheum. 2010; 62: 222-233. doi:10.1002/art.27233
5. Kennedy W P, Maciuca R, Wolslegel K, Tew W, Abbas A R, Chaivorapol C, et al. Association of the interferon signature metric with serological disease manifestations but not global activity scores in multiple cohorts of patients with SLE. Lupus Sci Med. 2015; 2: e000080. doi:10.1136/lupus-2014-000080
6. Rovin B H, Furie R, Latinis K, Looney R J, Fervenza F C, Sanchez-Guerrero J, et al. Efficacy and safety of rituximab in patients with active proliferative lupus nephritis: the Lupus Nephritis Assessment with Rituximab study. Arthritis Rheum. 2012;64: 1215-1226. doi:10.1002/art.34359
7. Banchereau R, Hong S, Cantarel B, Baldwin N, Baisch J, Edens M, et al. Personalized Immunomonitoring Uncovers Molecular Networks that Stratify Lupus Patients. Cell. Elsevier Inc.; 2016; 165: 551-565. doi:10.1016/j.cell.2016.03.008

1. A method for treating an individual with an autoimmune or inflammatory disease, comprising administering a therapeutically effective amount of a BTK inhibitor to the individual, wherein a sample from the individual has been found to have elevated levels of one or more biomarkers selected from the group consisting of IgJ, Mzb1, and Txndc5.

2. A method for treating an autoimmune or inflammatory disease in an individual, the method comprising:
(a) determining that a sample from the individual comprises elevated levels of one or more biomarkers selected from the group consisting of IgJ, Mzb1, and Txndc5; and
(b) administering an effective amount of a BTK inhibitor to the individual, whereby the autoimmune or inflammatory disease is treated.

3. A method for selecting a therapy for an individual with an autoimmune or inflammatory disease, comprising determining levels of one or more biomarkers selected from the group consisting of IgJ, Mzb1, and Txndc5; and selecting a medicament based on the levels of the one or more biomarkers.

4. A method of identifying an individual having an autoimmune or inflammatory disease who is more or less likely to exhibit benefit from treatment comprising a BTK inhibitor, comprising determining levels of one or more biomarkers selected from the group consisting of IgJ, Mzb1, and Txndc5 in a sample from the individual, wherein elevated levels of the one or more biomarkers in the sample indicates that the individual is more likely to exhibit benefit from treatment comprising the BTK inhibitor or reduced levels of the one or more biomarkers indicates that the individual is less likely to exhibit benefit from treatment comprising the BTK inhibitor.

5. An assay for identifying an individual with an autoimmune or inflammatory disease to receive a BTK inhibitor, the method comprising:
(a) determining levels of one or more biomarkers selected from the group consisting of IgJ, Mzb1, and Txndc5 in a sample from the individual; and
(b) recommending administration of a BTK inhibitor based upon the levels of the one or more biomarkers.

6. A diagnostic kit comprising one or more reagents for determining levels of one or more biomarkers selected from the group consisting of IgJ, Mzb1, and Txndc5 in a sample from an individual with an autoimmune or inflammatory disease, wherein detection of elevated levels of the one or more biomarkers means increased efficacy when the individual is treated with a BTK inhibitor, and wherein detection of low or substantially undetectable levels of the one or more biomarkers means a decreased efficacy when the individual with the autoimmune or inflammatory disease is treated with the BTK inhibitor.

7. The method of claim 3, wherein the method further comprises administering an effective amount of the BTK inhibitor to the individual.

8. The method of claim 1, wherein determining the levels of the biomarkers is performed by measuring RNA levels of the biomarkers relative to a reference level.

9. The method of claim 8, wherein measuring the RNA levels comprises amplification.

10. The method of claim 9, wherein measuring the RNA levels comprises quantitative PCR.

11. The method, assay and/or kit of claim 10, wherein measuring the RNA levels comprises amplifying the RNA and detecting the amplified product, thereby measuring the level of the RNA relative to a reference level.

12. The method of claim 1, wherein the sample is a blood sample.

13. The method of claim 1, wherein the BTK inhibitor is an antibody, binding polypeptide, small molecule, and/or polynucleotide.

14. The method of claim 13, wherein the BTK inhibitor is a small molecule.

15. The method of claim 14, wherein the small molecule is BTK inhibitor is Compound (A): 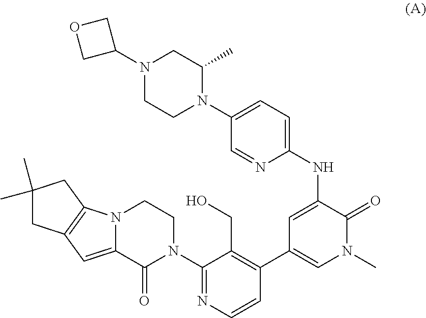 [SMILES: C[C@H]1CN(C2COC2)CCN1c1ccc(Nc2cc(-c3ccnc(N4CCn5c(cc6c5CC(C)(C)C6)C4=O)c3CO)cn(C)c2=O)nc1]
or a pharmaceutically acceptable salt thereof.

16. The method of claim 1, wherein the autoimmune or inflammatory disease is systemic lupus erythematosus.

17. The method of claim 16, wherein the autoimmune or inflammatory disease is lupus nephritis.

18. The method of claim 16, wherein the autoimmune or inflammatory disease is extra-renal lupus.

19. The method of claim 1, wherein two of the biomarkers are selected.

20. The method of claim 1, wherein three of the biomarkers are selected.
